(19) United States
(12) Patent Application Publication
(10) Pub. No.: US 2025/0376686 A1
(43) Pub. Date: Dec. 11, 2025

(54) MULTI-FUNCTIONAL NEAR-INFRARED FLUORESCENT POLYMER DOT-SIRNA FOR GENE EXPRESSION REGULATION

(71) Applicant: University of North Dakoa, Grand Forks, ND (US)

(72) Inventors: Di Sun, Grand Forks, ND (US); Julia Zhao, Grand Forks, ND (US); Diane Darland, Grand Forks, ND (US)

(21) Appl. No.: 19/235,302

(22) Filed: Jun. 11, 2025

Related U.S. Application Data
(60) Provisional application No. 63/658,687, filed on Jun. 11, 2024.

(51) Int. Cl. C12N 15/113; A61K 47/58; G01N 33/58

(52) U.S. Cl. CPC C12N 15/1135; A61K 47/58; G01N 33/582; C12N 2310/14; C12N 2310/3517; C12N 2310/51; C12N 2310/531

(57) ABSTRACT

A multi-functional near-infrared fluorescent polymer dot (Pdot)-siRNA nanoplatform is disclosed herein. The disclosed technology addresses challenges in siRNA delivery, including poor stability, degradation, and immune recognition, by utilizing positively charged Pdots synthesized from polymers, and electrostatic binding with negatively charged siRNA. The Pdots exhibit dual fluorescence emission at 588 nm and 775 nm, enabling real-time visualization of cellular uptake and siRNA delivery. The nanoplatform demonstrates efficient inhibition of target gene expression, and protein levels in cells. The Pdots provide minimal toxicity and persist in cells for extended periods, offering a robust tool for therapeutic applications, bioimaging, and molecular labeling. This approach combines siRNA delivery with simultaneous imaging, presenting a versatile method for targeted gene regulation and research applications.

### PRIORITY

This application claims the benefit of the filing date of U.S. provisional application No. 63/658,687, filed Jun. 11, 2024, the disclosure of which is incorporated by reference herein.

### GOVERNMENT SUPPORT

This invention was made with government support under 1709160 awarded by the National Science Foundation Division of Chemistry. The government has certain rights in the invention.

### INCORPORATION BY REFERENCE

This application contains a Sequence Listing which has been submitted electronically in ST26 format and hereby incorporated by reference in its entirety. Said ST26 file, created on Jun. 11, 2025, is named 3311037US1.xml and is 6,424 bytes in size.

### BACKGROUND OF THE INVENTION

Regulation of gene expression in eukaryotic cells plays a role in cell survival, proliferation, and cell fate determination. Mis-regulation of gene expression can have substantial, negative consequences that result in disease or tissue disfunction that can be targets for therapeutic intervention. A variety of strategies to inhibit gene expression at the level of mRNA transcription and translation have been developed. These include anti-sense inhibition, short interfering RNA, and CRISPR-Cas9 gene editing. However, there remain some limitations in the areas of specificity, toxicity, and ease-of-use for each of these approaches.

### SUMMARY

A nanomaterials-based tool is provided herein to inhibit gene expression in eukaryotic cells. Provided herein is a polymer-dot (Pdot-)-based platform that provides a means to deliver nucleic acids, such as inhibitory nucleic acids, including gene-specific siRNA into cells while at the same time providing a visualization mechanism to determine which cells have taken up the siRNA. These results highlight the application of the Pdot-siRNA for gene expression targeting with simultaneous visual monitoring of Pdot-siRNA delivery. The simple design offers a flexible and novel strategy to inhibit a wide range of mRNA targets with minimal toxicity, high efficiency, and focused cell visualization.

In one embodiment, the disclosure includes a polymer dot-nucleic acid composition comprising a polymer dot that includes poly[(2-methoxy-5-(2-ethylhexyloxy)-1,4-phenylenevinylene)], poly[(2,6-(4,4-bis(2-ethylhexyl)-4H-cyclopenta (2,1-b;3,4-b′)dithiophene)-alt-4,7 (2,1,3-benzothiadiazole))], and 1,2-distearoyl-sn-glycero-3-phosphoethanolamine-polyethylene glycol-polyetherimide, and one or more inhibitory nucleic acids bound to the polymer dot. In some embodiments, the polymer dot has a diameter of about 50 to about 100 nm, and the nucleic acid may be non-covalently bound to the polymer dot. In other embodiments, the polymer dot includes one or more functional groups selected from carboxylic acid, amino, mercapto, azido, alkyne, hydroxyl, and aldehyde groups, whereby the inhibitory nucleic acid is covalently bound. Certain embodiments further provide that a plurality of nucleic acid molecules are bound to the surface of the polymer dot, wherein the inhibitory nucleic acid is small interfering RNA having a length of at least 20 base pairs or about 20 to about 25 base pairs. In some cases, the polymer dot further comprises an additional therapeutic agent, such as a chemotherapeutic drug.

In another embodiment, the disclosure includes methods related to the use of the composition. One method involves regulating gene expression in a eukaryotic cell by contacting the cell with the composition and permitting uptake of the composition by the cell. In other embodiments, the composition is administered to a subject in need of treatment to inhibit gene expression, with particular applications to subjects having cancer where the inhibitory nucleic acid is specific for a cancer gene. Additionally, the disclosure provides methods to visualize nucleic acids within cells by contacting the cells with the composition and detecting the fluorescence emitted by the polymer dot.

### DRAWINGS

The drawings illustrate generally, by way of example, but not by way of limitation, various embodiments discussed herein.

FIGS. 1A-1B. Synthesis of DSPE-PEG-PEI amphiphilic polymer. (A) The synthetic route of DSPE-PEG-PEI polymer using starting materials including DSPE-PEG-NHS (MW 3,400) and PEI (MW 25,000) is shown. (B) 1H-NMR spectra of PEI, DSPE-PEG-NHS and DSPE-PEG-PEI is shown with the scale displayed in parts per million (ppm). Individual tracings are shown for each polymer component mix, and individual spectra are labeled with the ppm indicated for each. The 1H-NMR spectrum of DSPE-PEG-PEI in D2O exhibited characteristic peaks at 1.0-1.5 ppm (peaks of DSPE shown stearoyl tails), 2.5-3.0 ppm (peaks of PEI) and 3.7 ppm (peaks of PEG) indicating that PEI reacted with DSPE-PEG-NHS to form DSPE-PEG-PEI.

FIGS. 2A-2B. Formation of Pdots and Pdot-siRNA and a delivery mechanism to the cell. (A) The schematic diagram shows the synthesis of Pdots and binding with siRNA around the Pdots. The dashed box depicts a potential model for how the Pdots and siRNA are connected through electrostatic interactions. (B) Schematic illustration of the current working model regarding how the Pdot-siRNA nanoplatform may undergo cellular uptake and siRNA delivery to target transcripts.

FIGS. 3A-3H. Morphology investigation of Pdots and Pdot-siRNA in HEPES buffer. (A) TEM image, size distribution, and zeta potential of Pdots. (B) TEM image, size distribution, and zeta potential of Pdot-0.5 nmole control siRNA. (C) TEM image, size distribution, and zeta potential of Pdot-1.0 nmole control siRNA. (D) TEM image, size distribution, and zeta potential of Pdot-0.5 nmole control siRNA. (E) TEM image, size distribution, and zeta potential of Pdot-1.0 nmole Gapdh siRNA. (F) Average size comparison of Pdots and Pdot-siRNA. (G) Average zeta potential comparison of Pdots and Pdot-siRNA. (H) Average PDI comparison of Pdots and Pdot-siRNA. The red scale bar is 0.2 μm in A-E.

FIGS. 4A-4E. Absorbance properties of Pdots, Pdot-control siRNA, and Pdot-Gapdh siRNA. (A) Absorbance properties of Pdots under a concentration series from 1 μg/mL to 10 μg/mL. (B) Linear regression analysis of different concentrations of Pdots versus absorbance peak at 500 nm. (C) Linear regression analysis of different concentrations of Pdots versus absorbance peak at 693 nm. (D) Absorbance property of Pdots with a certain amount (0.5 nmole and 1.0 nmole) of control siRNA. (E) Absorbance property of Pdots with a certain amount (0.5 nmole and 1.0 nmole) of Gapdh siRNA.

FIGS. 5A-5D. Fluorescence properties of Pdots in a dose series from 1 μg/mL to 10 μg/mL. (A) Fluorescent spectra of Pdots through fix excitation at 500 nm and emission at 588 nm. (B) Linear regression analysis of a series concentrations of Pdots versus emission fluorescence intensity at 588 nm while excitation was fixed at 500 nm. (C) Linear regression analysis of a concentration series of Pdots versus emission fluorescence intensity at 775 nm with excitation fixed at 500 nm. (D) Fluorescent activity of Pdots alone or when coupled to either control or Gapdh siRNA.

FIG. 6. Imaging of BMVFb cells incubated with Pdots for 4 h. BMVFb were cultured in 8-well Lab Tek Chamber slides at a target plating density of 2,500 cells/well, treated with different concentrations of Pdots (red) for 4 h and then post-fixation labeled with DAPI (blue, nuclei) and Alexa Fluor 488 phalloidin (green, actin). The red arrows indicated healthy cells with normal-sized, round nuclei while the white arrows indicate an intermediate stage of cell that is likely undergoing cell death (small, round nuclei) or is already pyknotic. The yellow circles indicated the perinuclear Pdots fluorescent signal. The scale bar is 50 μm for all panels and the three-channel overlay is shown for comparison with the individual channels.

FIG. 7. Imaging of BMVFb cells incubated with Pdots for 24 h. BMVFb were cultured as described for FIG. 6 but were incubated with varying Pdots concentrations for 24 h and then post-fixation labeled with DAPI (blue, nuclei) and Alexa Fluor 488 phalloidin (green, actin). The red arrows indicated that there were still healthy cells with normal-sized and round nuclei after treatment with 5 μg/mL of Pdots. The scale bar is 50 μm in all panels.

FIGS. 8A-8D. Summary of cytotoxicity under different incubation times with different concentrations of Pdots on BMVFb cells. Pdots were incubated with the cells for 4 h (A), 8 h (B), 12 h (C), or 24 h (D) at the concentrations indicated on the x-axes. Data are presented as the mean±SD, n=3. The one-way ANOVA showed significant differences comparing the highest concentration of Pdots (20 μg/mL) with the lower concentration of Pdots and control, although cell viability for all concentrations closely matched controls. The pair-wise analysis with Tukey multiple comparisons post hoc test showed significance with bars indicating a comparison: p<0.01 was presented as **, p<0.001 was presented as ***, and p<0.0001 was presented as ****.

FIG. 9. BMVEC incubated with Pdots and Pdots with different amounts of control siRNA for 4 h. The cells were fixed and labeled with DAPI (blue nuclei). The green channel was a fluorescent signal from Pdots, with a set detection range from 550-650 nm. The red channel was a fluorescent signal from Pdots, with a set detection range from 700-800 nm. The scale bar was 10 μm.

FIG. 10. Gapdh expression is reduced in BMVEC treated with Pdot-Gapdh siRNA. Expression of Gapdh was assessed via qPCR in BMVEC treated with control (Pdots alone, siRNA alone or Pdot-siRNA-ctl) or target-specific Pdot-siRNA-Gapdh. The mean and standard deviation are graphed for the 2\hat{ }-ΔΔ C_{T} values of n=3 or 4 samples per treatment condition. Cells were treated for 24 h prior to RNA collection. One-way ANOVA results indicated that the means were significantly different (p=0.0373) with F=3.071, DFn=5, and DFd=17. Dunnett's Multiple Comparisons test of each population relative to Pdots alone as the control indicated significance for the Pdot-siRNA-Gapdh (0.5 nmoles) with a set at 0.05 and indicated by “*”.

FIG. 11. Gapdh immunolabeling has lower intensity in BMVEC treated with Pdot-Gapdh siRNA compared to Pdots and Pdot-Control siRNA. The BMVEC were labeled with Gapdh antibody (green channel) and with DAPI (blue nuclei) across all treatment conditions. The fluorescent signal from Pdots was detected in two emission ranges (red-violet channel; 550-650 and red channel; 700-800 nm) as expected. The overlay of all channels is shown in the bottom panels for comparison. The scale bar is 50 μm.

FIGS. 12A-12C. Absorbance characterization of Pdots and Pdot-Control siRNA in water, biocompatible buffer (HEPES & PBS), and cell culture medium (DMEM). (A) The absorbance investigation of water, HEPES buffer, PBS buffer and DMEM medium. (B) The absorbance properties of Pdots in water, HEPES buffer, PBS buffer and DMEM medium. (C) The absorbance properties of Pdot-Inmole Control siRNA in water, HEPES buffer, PBS buffer and DMEM medium.

FIG. 13. Summary of Pdots and Pdot-Control siRNA in terms of size, zeta potential, PDI and conductivity in water, HEPES buffer, PBS buffer and DMEM medium. The test revealed that the nanomaterial average diameter differed in buffer versus cell culture media.

FIGS. 14A-14D. Fluorescence properties of Pdots in a dose series from 1 μg/mL to 10 μg/mL. (A) Overall fluorescent spectra of Pdots through fixed excitation at 500 nm and 693 nm and fixed emission at 588 nm and 775 nm. (B) Fluorescent spectra by fixed excitation at 500 nm and emission at 775 nm. (C) Linear regression analysis of different concentrations of Pdots versus excitation fluorescence intensity at 500 nm with emission fixed at 588 nm. (D) Linear regression analysis of different concentrations of Pdots versus excitation fluorescence intensity at 500 nm emission fixed at 775 nm.

FIGS. 15A-15C. Fluorescence properties of Pdots, Pdot-control siRNA and Pdot-Gapdh siRNA. (A) Fluorescence properties of Pdot-0.5 nmole control siRNA, Pdot-1.0 nmole control siRNA, Pdot-0.5 nmole Gapdh siRNA, and Pdot-1.0 nmole Gapdh siRNA were compared with Pdots alone. (B) Fluorescence spectra of Pdot-control siRNA and Pdot-Gapdh siRNA with excitation fixed at 500 nm and emission at 775 nm. Fluorescence at 500 nm and 775 nm had a similar intensity. (C) Fluorescence spectra of Pdot-control siRNA and Pdot-Gapdh siRNA is shown with the excitation wavelength fixed at 693 nm and emission fixed at 588 nm.

FIG. 16. Imaging of BMVFb cells incubated with Pdots for 8 h. BMVFb were cultured in 8-well Lab Tek Chamber slides at a target plating density of 2,500 cells/well, treated with different concentrations of Pdots (red) for 8 h and then post-fixation labeled with DAPI (blue, nuclei) and Alexa Fluor 488 phalloidin (green, actin). The white arrows highlight intermediate cells that are likely undergoing apoptosis and have small, pyknotic nuclei. The scale bar is 50 μm for all panels and the three-channel overlay is shown for comparison with the individual channels.

FIG. 17. Imaging of BMVFb cells incubated with Pdots for 12 h. BMVFb were cultured as described for FIG. 15 but were incubated with varying Pdots concentrations for 12 h and then post-fixation labeled with DAPI (blue, nuclei) and Alexa Fluor 488 phalloidin (green, actin). The white arrows in the figure (10 μg/mL of Pdots) identify cells with small, round nuclei. The scale bar is 50 μm for all panels.

FIG. 18. Pdots' signal persistence in BMVFb cells. The cells were incubated with 10 μg/mL Pdots for 4 h, and the live cells were further incubated for the time points as indicated. The chamber slides were fixed and labeled with DAPI (blue, nuclei) and Alexa Fluor 488 phalloidin (green, actin). The scale bar is 50 μm.

FIG. 19. Primer sequence information, including full gene name, NCBI accession number, sequence (5′ to 3′) (SEQ ID NOs: 1-6), and amplicon size in base pairs (bp).

Unless otherwise indicated, all figures and drawings in this document are not to scale and are chosen for the purpose of illustrating different embodiments of the invention. The dimensions of the various components are depicted in illustrative terms only, and no relationship between the dimensions of the various components should be inferred from the drawings, unless so indicated. Although terms such as “top”, “bottom”, “upper”, “lower”, “under”, “over”, “front”, “back”, “up” and “down”, and “first” and “second” can be used in this disclosure, it should be understood that those terms are used in their relative sense only unless otherwise noted.

### DESCRIPTION

Reference will now be made in detail to certain embodiments of the disclosed subject matter. While the disclosed subject matter will be described in conjunction with the enumerated claims, it will be understood that the exemplified subject matter is not intended to limit the claims to the disclosed subject matter.

The regulation of gene expression in eukaryotic cells is a biological process that plays a significant role in influencing cell survival, proliferation, and differentiation. Misregulation of gene expression can result in severe consequences, including disease and tissue dysfunction, which are often addressed through therapeutic intervention. Conventional methods for inhibiting gene expression, such as anti-sense inhibition, short interfering RNA (siRNA), and CRISPR-Cas9 gene editing, have shown promise but face notable challenges. These include difficulties in achieving high specificity, reducing toxicity, and ensuring ease of use. Furthermore, the delivery of siRNA to target cells remains a substantial challenge due to its poor stability in circulation, vulnerability to enzymatic degradation, and recognition by the immune system, which collectively diminish its therapeutic effectiveness.

The present disclosure addresses these limitations by introducing a multi-functional near-infrared fluorescent polymer dot (Pdot)-siRNA nanoplatform for gene expression regulation. This approach leverages the distinctive characteristics of polymer dots synthesized from polymers. The Pdots are positively charged, enabling electrostatic binding with negatively charged siRNA, which enhances stability and protects the siRNA from enzymatic degradation. Furthermore, the Pdots exhibit dual fluorescence emission at 588 nm and 775 nm, allowing real-time visualization of cellular uptake and siRNA delivery. This dual functionality not only facilitates effective delivery of siRNA to target cells but also provides a mechanism for monitoring the delivery process, addressing the specificity and visualization challenges commonly associated with conventional methods.

By combining siRNA delivery with simultaneous imaging capabilities, the disclosed nanoplatform offers a robust and versatile tool for targeted gene regulation. The disclosed technology demonstrates minimal toxicity at optimized concentrations, efficient inhibition of target gene expression, and extended persistence in cells, as validated through experimental studies. This approach represents a significant advancement over prior methods, providing a flexible, efficient, and low-toxicity solution for therapeutic applications, bioimaging, and molecular labeling.

### Definitions

The following definitions are included to provide a clear and consistent understanding of the specification and claims. As used herein, the recited terms have the following meanings. All other terms and phrases used in this specification have their ordinary meanings as one of skill in the art would understand. Such ordinary meanings may be obtained by reference to technical dictionaries, such as Hawley's Condensed Chemical Dictionary 14th Edition, by R. J. Lewis, John Wiley & Sons, New York, N. Y., 2001.

References in the specification to “one embodiment,” “an embodiment,” etc., indicate that the embodiment described may include a particular aspect, feature, structure, moiety, or characteristic, but not every embodiment necessarily includes that aspect, feature, structure, moiety, or characteristic. Moreover, such phrases may, but do not necessarily, refer to the same embodiment referred to in other portions of the specification. Further, when a particular aspect, feature, structure, moiety, or characteristic is described in connection with an embodiment, it is within the knowledge of one skilled in the art to affect or connect such aspect, feature, structure, moiety, or characteristic with other embodiments, whether or not explicitly described.

The singular forms “a,” “an,” and “the” include plural reference unless the context clearly dictates otherwise. Thus, for example, a reference to “a compound” includes a plurality of such compounds, so that a compound X includes a plurality of compounds X. It is further noted that the claims may be drafted to exclude any optional element. As such, this statement is intended to serve as antecedent basis for the use of exclusive terminology, such as “solely,” “only,” and the like, in connection with any element described herein, and/or the recitation of claim elements or use of “negative” limitations.

The term “and/or” means any one of the items, any combination of the items, or all of the items with which this term is associated. The phrase “one or more” is readily understood by one of skill in the art, particularly when read in context of its usage. For example, one or more substituents on a phenyl ring refers to one to five, or one to four, for example if the phenyl ring is di-substituted.

As used herein, “or” should be understood to have the same meaning as “and/or” as defined above. For example, when separating a listing of items, “and/or” or “or” shall be interpreted as being inclusive, e.g., the inclusion of at least one, but also including more than one of a number of items, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of.”

As used herein, the terms “including,” “includes,” “having,” “has,” “with,” or variants thereof, are intended to be inclusive similar to the term “comprising.”

The term “about” can refer to a variation of +5%, +10%, +20%, or +25% of the value specified. For example, “about 50” percent can in some embodiments carry a variation from 45 to 55 percent. For integer ranges, the term “about” can include one or two integers greater than and/or less than a recited integer at each end of the range. Unless indicated otherwise herein, the term “about” is intended to include values, e.g., weight percentages, proximate to the recited range that are equivalent in terms of the functionality of the individual ingredient, the composition, or the embodiment. The term about can also modify the endpoints of a recited range as discuss above in this paragraph.

As will be understood by the skilled artisan, all numbers, including those expressing quantities of ingredients, properties such as molecular weight, reaction conditions, and so forth, are approximations and are understood as being optionally modified in all instances by the term “about.” These values can vary depending upon the desired properties sought to be obtained by those skilled in the art utilizing the teachings of the descriptions herein. It is also understood that such values inherently contain variability necessarily resulting from the standard deviations found in their respective testing measurements.

As will be understood by one skilled in the art, for any and all purposes, particularly in terms of providing a written description, all ranges recited herein also encompass any and all possible sub-ranges and combinations of sub-ranges thereof, as well as the individual values making up the range, particularly integer values. A recited range (e.g., weight percentages or carbon groups) includes each specific value, integer, decimal, or identity within the range. Any listed range can be easily recognized as sufficiently describing and enabling the same range being broken down into at least equal halves, thirds, quarters, fifths, or tenths. As a non-limiting example, each range discussed herein can be readily broken down into a lower third, middle third and upper third, etc. As will also be understood by one skilled in the art, all language such as “up to,” “at least,” “greater than,” “less than,” “more than,” “or more,” and the like, include the number recited and such terms refer to ranges that can be subsequently broken down into sub-ranges as discussed above. In the same manner, all ratios recited herein also include all sub-ratios falling within the broader ratio. Accordingly, specific values recited for radicals, substituents, and ranges, are for illustration only; they do not exclude other defined values or other values within defined ranges for radicals and substituents.

One skilled in the art will also readily recognize that where members are grouped together in a common manner, such as in a Markush group, the invention encompasses not only the entire group listed as a whole, but each member of the group individually and all possible subgroups of the main group.

Additionally, for all purposes, the invention encompasses not only the main group, but also the main group absent one or more of the group members. The invention therefore envisages the explicit exclusion of any one or more of members of a recited group. Accordingly, provisos may apply to any of the disclosed categories or embodiments whereby any one or more of the recited elements, species, or embodiments, may be excluded from such categories or embodiments, for example, for use in an explicit negative limitation.

The term “contacting” refers to the act of touching, making contact, or of bringing to immediate or close proximity, including at the cellular or molecular level, for example, to bring about a physiological reaction, a chemical reaction, or a physical change, e.g., in a solution, in a reaction mixture, in vitro, or in vivo.

An “effective amount” refers to an amount effective to treat a disease, disorder, and/or condition, or to bring about a recited effect. For example, an effective amount can be an amount effective to reduce the progression or severity of the condition or symptoms being treated. Determination of a therapeutically effective amount is well within the capacity of persons skilled in the art, especially in light of the detailed disclosure provided herein. The term “effective amount” is intended to include an amount of a compound described herein, or an amount of a combination of compounds described herein, e.g., that is effective to treat or prevent a disease or disorder, or to treat the symptoms of the disease or disorder, in a host. Thus, an “effective amount” generally means an amount that provides the desired effect.

The terms “treating,” “treat” and “treatment” include (i) preventing a disease, pathologic or medical condition from occurring (e.g., prophylaxis); (ii) inhibiting the disease, pathologic or medical condition or arresting its development; (iii) relieving the disease, pathologic or medical condition; and/or (iv) diminishing symptoms associated with the disease, pathologic or medical condition. Thus, the terms “treat”, “treatment”, and “treating” can extend to prophylaxis and can include prevent, prevention, preventing, lowering, stopping or reversing the progression or severity of the condition or symptoms being treated. As such, the term “treatment” can include medical, therapeutic, and/or prophylactic administration, as appropriate.

The use of the word “detect” and its grammatical variants refers to measurement of the species without quantification, whereas use of the word “determine” or “measure” with their grammatical variants are meant to refer to measurement of the species with quantification. The terms “detect” and “identify” are used interchangeably herein.

The term “nucleic acid” typically refers to large polynucleotides. By “nucleic acid” is meant any nucleic acid, whether composed of deoxyribonucleosides or ribonucleosides, and whether composed of phosphodiester linkages or modified linkages such as phosphotriester, phosphoramidate, siloxane, carbonate, carboxymethylester, acetamidate, carbamate, thioether, bridged phosphoramidate, bridged methylene phosphonate, bridged phosphoramidate, bridged phosphoramidate, bridged methylene phosphonate, phosphorothioate, methylphosphonate, phosphorodithioate, bridged phosphorothioate or sulfone linkages, and combinations of such linkages. The term nucleic acid also specifically includes nucleic acids composed of bases other than the five biologically occurring bases (adenine, guanine, thymine, cytosine and uracil).

As used herein, the term “nucleic acid” encompasses RNA as well as single and double stranded DNA and cDNA. Furthermore, the terms, “nucleic acid,” “DNA,” “RNA” and similar terms also include nucleic acid analogs, i.e., analogs having other than a phosphodiester backbone. For example, the so called “peptide nucleic acids,” which are known in the art and have peptide bonds instead of phosphodiester bonds in the backbone, are considered within the scope of the present invention. By “nucleic acid” is meant any nucleic acid, whether composed of deoxyribonucleosides or ribonucleosides, and whether composed of phosphodiester linkages or modified linkages such as phosphotriester, phosphoramidate, siloxane, carbonate, carboxymethylester, acetamidate, carbamate, thioether, bridged phosphoramidate, bridged methylene phosphonate, bridged phosphoramidate, bridged phosphoramidate, bridged methylene phosphonate, phosphorothioate, methylphosphonate, phosphorodithioate, bridged phosphorothioate or sulfone linkages, and combinations of such linkages. The term nucleic acid also specifically includes nucleic acids composed of bases other than the five biologically occurring bases (adenine, guanine, thymine, cytosine, and uracil). Conventional notation is used herein to describe polynucleotide sequences: the left-hand end of a single-stranded polynucleotide sequence is the 5′-end; the left-hand direction of a double-stranded polynucleotide sequence is referred to as the 5′-direction. The direction of 5′ to 3′ addition of nucleotides to nascent RNA transcripts is referred to as the transcription direction. The DNA strand having the same sequence as an mRNA is referred to as the “coding strand”; sequences on the DNA strand which are located 5′ to a reference point on the DNA are referred to as “upstream sequences”; sequences on the DNA strand which are 3′ to a reference point on the DNA are referred to as “downstream sequences.”

The term “oligonucleotide” typically refers to short polynucleotides, generally, no greater than about 50 nucleotides. It will be understood that when a nucleotide sequence is represented by a DNA sequence (i.e., A, T, G, C), this also includes an RNA sequence (i.e., A, U, G, C) in which “U” replaces “T.”

As used herein, the term “pharmaceutically acceptable carrier” means a chemical composition with which an appropriate compound or derivative can be combined and which, following the combination, can be used to administer the appropriate compound to a subject. “Pharmaceutically acceptable” means physiologically tolerable, for either human or veterinary application. As used herein, “pharmaceutical compositions” include formulations for human and veterinary use. Administration can be by various means, including systemic and local injection.

The term “regulate” refers to either stimulating or inhibiting a function or activity of interest.

The term “standard,” as used herein, refers to something used for comparison. For example, it can be a known standard agent or compound which is administered and used for comparing results when administering a test compound, or it can be a standard parameter or function which is measured to obtain a control value when measuring an effect of an agent or compound on a parameter or function. Standard can also refer to an “internal standard”, such as an agent or compound which is added at known amounts to a sample and is useful in determining such things as purification or recovery rates when a sample is processed or subjected to purification or extraction procedures before a marker of interest is measured. Internal standards are often a purified marker of interest which has been labeled, such as with a radioactive isotope, allowing it to be distinguished from an endogenous marker.

As used herein, a “subject in need thereof” is a patient, animal, mammal, or human, who will benefit from the method of this invention.

Cancer is a group of diseases involving abnormal cell growth with the potential to invade or spread to other parts of the body. There are numerous types of cancer, broadly classified by the tissue or organ where they originate. These include carcinomas, sarcomas, leukemias, lymphomas, and melanomas. Carcinomas, the most common type, originate in the skin or tissues lining internal organs, while sarcomas develop in connective and supportive tissues like bone and muscle. Leukemias and lymphomas are cancers of blood cells and lymphatic tissues, respectively. Types include breast cancer, lung cancer, colon cancer, pancreatic cancer, kidney cancer, bladder cancer, brain tumor, neuroblastoma, bone cancer, soft tissue sarcoma, acute lymphoblastic leukemia and acute myeloid leukemia. Hodgkin lymphoma and non-Hodgkin lymphoma and melanoma.

Methods involving conventional molecular biology techniques are described herein. Such techniques are generally known in the art and are described in detail in methodology treatises, such as Molecular Cloning: A Laboratory Manual, 2nd ed., vol. 1-3, ed. Sambrook et al., Cold Spring Harbor Laboratory Press, Cold Spring Harbor, N.Y., 1989; and Current Protocols in Molecular Biology, ed. Ausubel et al., Greene Publishing and Wiley-Interscience, New York, 1992 (with periodic updates). Methods for chemical synthesis of nucleic acids are discussed, for example, in Beaucage and Carruthers, Tetra. Letts. 22:1859-1862, 1981, and Matteucci et al., J. Am. Chem. Soc. 103:3185, 1981.

As used herein, the term “polymer dot” refers to a structure comprising one or more polymers. The nanoparticles provided herein may be formed by any method known in the art, including without limitation, methods relying on precipitation (including nanoprecipitation), methods relying on the formation of emulsions (e.g., mini or micro emulsion), and methods relying on condensation.

As used herein, “polymer” is a molecule composed of at least 2 repeating structural units typically connected by covalent chemical bonds. Polymers generally have extended molecular structures comprising backbones that optionally contain pendant side groups. It includes linear polymer and branched polymer such as star polymers, comb polymers, brush polymers, ladders, and dendrimers.

As used herein, the term “functional group” refers to any chemical unit that can be attached, such as by any stable physical or chemical association, to the polymer, thereby rendering the surface of the polymer dot available for conjugation. Non-limiting examples of functional groups include, carboxylic acid, amino, mercapto, azido, alkyne, aldehyde, hydroxyl, carbonyl, sulfate, sulfonate, phosphate, cyanate, succinimidyl ester, alkyne, strained alkyne, azide, diene, alkene, cyclooctyne, and phosphine groups, substituted derivatives thereof, and combinations thereof.

As used herein the term “hydrophilic functional group” refers either to a functional group that is hydrophilic in nature or to a hydrophobic functional group that is attached to a hydrophilic side chain or hydrophilic moiety, which renders the hydrophobic functional group more hydrophilic in nature and which facilitate the arrangement of the hydrophobic functional groups on the polymer dot particle surface rather than getting buried inside the hydrophobic core of the polymer dot. Examples of hydrophobic functional groups that can be rendered more hydrophilic by attachment to hydrophilic side chains or moieties include but not limited to alkyne, strained alkyne, azide, diene, alkene, cyclooctyne, and phosphine groups (for click chemistry) attached to a hydrophilic side chain such as PEG (polyethylene glycol) or to any other hydrophilic side chains.

As used herein, the term “bioorthogonal reaction” refers to a conjugation between non-native, non-perturbing chemical handles that can be modified in living systems through highly selective reactions with exogenously delivered probes. The most well-known of the bioorthogonal reaction schemes is known as click chemistry. For review of bioorthogonal reaction schemes, see, for example Best M D, Biochemistry. 2009 Jul. 21; 48 (28): 6571-84, the disclosure of which is herein incorporated by reference in its entirety for all purposes.

As used herein, the term “click reaction” is recognized in the art, which describe a collection of reliable and self-directed organic reactions, such as the most recognized copper catalyzed azide-alkyne [3+2] cycloaddition. Non-limiting examples of click chemistry reactions can be found, for example, in H. C. Kolb, M. G. Finn, K. B. Sharpless, Angew. Chem. Int. Ed. 2001, 40, 2004 and E. M. Sletten, C. R. Bertozzi, Angew. Chem. Int. Ed. 2009, 48, 6974, the disclosures of which are herein incorporated by reference in their entireties for all purposes.

As used herein, the term “cross-linking agent” is used to describe a compound that is capable of forming a chemical bond between molecular groups on similar or dissimilar molecules so as to covalently bond together the molecules. Examples of common cross-linking agents are known in the art. See, for example, Bioconjugate Techniques (Academic Press, New York, 1996 or later versions) the content of which is herein incorporated by reference in its entirety for all purposes. Indirect attachment of the biomolecule to polymer dots can occur through the use of “linker” molecule, for example, avidin, streptavidin, neutravidin, biotin or a like molecule.

By “small interfering RNAs (siRNAs)” is meant, inter alia, an isolated dsRNA molecule comprised of both a sense and an anti-sense strand. In one aspect, it is greater than 10 nucleotides in length. siRNA also refers to a single transcript which has both the sense and complementary antisense sequences from the target gene, e.g., a hairpin. siRNA further includes any form of dsRNA (proteolytically cleaved products of larger dsRNA, partially purified RNA, essentially pure RNA, synthetic RNA, recombinantly produced RNA) as well as altered RNA that differs from naturally occurring RNA by the addition, deletion, substitution, and/or alteration of one or more nucleotides.

### Polymer Dots

The present invention provides, in one aspect, a polymer dot (Pdot).

In one embodiment, the polymer dots comprise, consist of or consist essentially of a blend of poly[2-methoxy-5-(2-ethylhexyloxy)-1,4-phenylenevinylene] (MEHPPV), Poly[2,6-(4,4-bis-(2-ethylhexyl)-4H-cyclopenta[2,1-b;3,4-b′]dithiophene)-alt-4,7 (2,1,3-benzothiadiazole)] (PCPDTBD) and/or 1,2-distearoyl-sn-glycero-3-phosphoethanolamine-polyethylene glycol-polyetherimide (DSPE-PEG-PEI).

### Bio-Conjugated Polymer Dots

In one aspect, the present invention provides a bioconjugate comprising a polymer dot as described herein and a biomolecule, wherein the biomolecule is attached to the polymer dot either directly or indirectly by any suitable means.

The term “biomolecule” is used to describe a synthetic or naturally occurring protein, glycoprotein, peptide, amino acid, metabolite, drug, toxin, nuclear acid, nucleotide, carbohydrate, sugar, lipid, fatty acid and the like. The biomolecule may be attached to the polymer dot directly or indirectly by any suitable means, such as by any stable physical or chemical association.

In one embodiment, the biomolecule is nucleic acid. In one embodiment, the nucleic acid is an inhibitory nucleic acid. In one embodiment, the nucleic acid is an RNA. In one embodiment, the nucleic acid is a small interfering RNA (siRNA), such as one that can inhibit gene expression.

The siRNA for use in the methods of the present invention can be synthesized or obtained from commercial sources. In one embodiment, the siRNA is double stranded and can comprise a sequence that is from about 19 nucleotides to about 30 nucleotides. In particular embodiments, the siRNA is double stranded and can comprise a sequence that is from about 21 nucleotides to about 27 nucleotides, or from about 23 to about 25 nucleotides. In other embodiments, the siRNA is double stranded and one or both strands (e.g., sense, antisense) can comprises a sequence of about 19, 20, 21, 22, 23, 24, 25, 26, 27, 28, 29, 30, 31, 32, 33, 34 or 35 nucleotides. The siRNA is comprised of RNA, and in some embodiments, can include DNA base pairs, either at the end of or within one or more of the strands of the siRNA.

The siRNA can comprise one or more blunt ends and/or one or more overhangs. Preparation of siRNA that comprise one or more blunt ends and/or one or more overhangs can be prepared using known in the art. The overhangs can be present on one or more strands of a double stranded siRNA. In one embodiment, an overhang can comprise from about 1 to about 5 nucleotides. In another embodiment, the overhand comprises 1, 2, 3, 4 or 5 nucleotides. The overhang can comprise RNA, and in some embodiments, DNA base pairs.

As used herein, the “target RNA sequence” is any RNA sequence in a cell or in an individual that is selected to be degraded using RNA interference. Many such sequences are known in the art. For example, the RNA sequence can be a viral sequence or a sequence of a protein that is associated with a cancer, such as an oncoprotein. There can also be a “target DNA sequence.”

### Inhibitory Nucleic Acids

The expression of one or more proteins can be inhibited, for example by use of an inhibitory nucleic acid that specifically recognizes a nucleic acid that encodes the protein.

An inhibitory nucleic acid can have at least one segment that will hybridize to a nucleic acid of interest under intracellular conditions. The inhibitory nucleic acid can reduce expression of nucleic acid of interest. A nucleic acid may hybridize to a genomic DNA, a messenger RNA, or a combination thereof. An inhibitory nucleic acid may be incorporated into a plasmid vector or viral DNA, or it may not be. It may be single stranded or double stranded, circular or linear.

An inhibitory nucleic acid is a polymer of ribose nucleotides or deoxyribose nucleotides having more than 13 nucleotides in length. An inhibitory nucleic acid may include naturally occurring nucleotides; synthetic, modified, or pseudo-nucleotides such as phosphorothiolates; as well as nucleotides having a detectable label such as P³², biotin or digoxigenin. An inhibitory nucleic acid can reduce the expression and/or activity of a nucleic acid interest. Such an inhibitory nucleic acid may be completely complementary to a segment of an endogenous nucleic acid (e.g., an RNA). Alternatively, some variability is permitted in the inhibitory nucleic acid sequences relative to the sequences of interest (e.g., pathway inhibitor). An inhibitory nucleic acid can hybridize to a nucleic acid of interest under intracellular conditions or under stringent hybridization conditions and is sufficiently complementary to inhibit expression of the endogenous nucleic acid of interest. Intracellular conditions refer to conditions such as temperature, pH and salt concentrations typically found inside a cell, e.g., an animal or mammalian cell. One example of such an animal or mammalian cell is a cancer cell. Generally, stringent hybridization conditions are selected to be about 5° C. lower than the thermal melting point (Tm) for the specific sequence at a defined ionic strength and pH. However, stringent conditions encompass temperatures in the range of about 1° C. to about 20° C. lower than the thermal melting point of the selected sequence, depending upon the desired degree of stringency as otherwise qualified herein. Inhibitory oligonucleotides that comprise, for example, 2, 3, 4, or 5 or more stretches of contiguous nucleotides that are precisely complementary to the coding sequence of interest, each separated by a stretch of contiguous nucleotides that are not complementary to adjacent coding sequences, can inhibit the function of one or more nucleic acids. In general, each stretch of contiguous nucleotides is at least 4, 5, 6, 7, or 8 or more nucleotides in length. Non-complementary intervening sequences may be 1, 2, 3, or 4 nucleotides in length. One skilled in the art can easily use the calculated melting point of an inhibitory nucleic acid hybridized to a sense nucleic acid to estimate the degree of mismatching that will be tolerated for inhibiting expression of a particular target nucleic acid. Inhibitory nucleic acids of the invention include, for example, a short hairpin RNA, a small interfering RNA, a ribozyme or an antisense nucleic acid molecule.

The inhibitory nucleic acid molecule may be single or double stranded (e.g., a small interfering RNA (siRNA)) and may function in an enzyme-dependent manner or by steric blocking. Inhibitory nucleic acid molecules that function in an enzyme-dependent manner include forms dependent on RNase H activity to degrade target mRNA. These include single-stranded DNA, RNA, and phosphorothioate molecules, as well as the double-stranded RNAi/siRNA system that involves target mRNA recognition through sense-antisense strand pairing followed by degradation of the target mRNA by the RNA-induced silencing complex. Steric blocking inhibitory nucleic acids, which are RNase-H independent, interfere with gene expression or other mRNA-dependent cellular processes by binding to a target mRNA and getting in the way of other processes. Steric blocking inhibitory nucleic acids include 2′-O alkyl (usually in chimeras with RNase-H dependent antisense), peptide nucleic acid (PNA), locked nucleic acid (LNA) and morpholino antisense.

Small interfering RNAs, for example, may be used to specifically reduce translation of a target nucleic acid such that translation of the encoded target polypeptide is reduced. siRNAs mediate post-transcriptional gene silencing in a sequence-specific manner. See, for example, website at invitrogen.com/site/us/en/home/Products-and-Services/Applications/rnai.html. Once incorporated into an RNA-induced silencing complex, siRNA mediate cleavage of the homologous endogenous mRNA transcript by guiding the complex to the homologous mRNA transcript, which is then cleaved by the complex. The siRNA may be homologous and/or complementary to any region of the target transcript. The region of homology may be 30 or 40 nucleotides or less in length, such less than 25 nucleotides, and such as about 21 to 23 nucleotides in length. SiRNA is typically double stranded and may have two-nucleotide 3′ overhangs, for example, 3′ overhanging UU dinucleotides. Methods for designing siRNAs are known to those skilled in the art. See, for example, Elbashir et al. Nature 411:494-498 (2001); Harborth et al. Antisense Nucleic Acid Drug Dev. 13:83-106 (2003).

The pSuppressorNeo vector for expressing hairpin siRNA, commercially available from IMGENEX (San Diego, California), can be used to generate siRNA for inhibiting expression of targets. The construction of the siRNA expression plasmid involves the selection of the target region of the mRNA, which can be a trial-and-error process. However, Elbashir et al. have provided guidelines that appear to work ˜80% of the time. Elbashir, S. M., et al., Analysis of gene function in somatic mammalian cells using small interfering RNAs. Methods, 2002. 26 (2): p. 199-213. Accordingly, for synthesis of synthetic siRNA, a target region may be selected about 50 to 100 nucleotides downstream of the start codon. The 5′ and 3′ untranslated regions and regions close to the start codon should be avoided as these may be richer in regulatory protein binding sites. As siRNA can begin with AA, have 3′ UU overhangs for both the sense and antisense siRNA strands, and have an approximate 50% G/C content. An example of a sequence for a synthetic siRNA is 5′-AA (N19) UU, where N is any nucleotide in the mRNA sequence and should be approximately 50% G-C content. The selected sequence(s) can be compared to others in the human genome database to minimize homology to other known coding sequences (e.g., by Blast search, for example, through the NCBI website).

siRNAs may be chemically synthesized, created by in vitro transcription, or expressed from an siRNA expression vector or a PCR expression cassette. See, e.g., website at invitrogen.com/site/us/en/home/Products-and-Services/Applications/rnai.html. When an siRNA is expressed from an expression vector or a PCR expression cassette, the insert encoding the siRNA may be expressed as an RNA transcript that folds into an siRNA hairpin. Thus, the RNA transcript may include a sense siRNA sequence that is linked to its reverse complementary antisense siRNA sequence by a spacer sequence that forms the loop of the hairpin as well as a string of U's at the 3′ end. The loop of the hairpin may be of any appropriate lengths, for example, 3 to 30 nucleotides in length, preferably, 3 to 23 nucleotides in length, and may be of various nucleotide sequences including, AUG, CCC, UUCG, CCACC, CTCGAG, AAGCUU, CCACACC and UUCAAGAGA. SiRNAs also may be produced in vivo by cleavage of double-stranded RNA introduced directly or via a transgene or virus. Amplification by an RNA-dependent RNA polymerase may occur in some organisms.

An inhibitory nucleic acid such as a short hairpin RNA siRNA or an antisense oligonucleotide may be prepared using methods such as by expression from an expression vector or expression cassette that includes the sequence of the inhibitory nucleic acid. Alternatively, it may be prepared by chemical synthesis using naturally occurring nucleotides, modified nucleotides or any combinations thereof. In some embodiments, the inhibitory nucleic acids are made from modified nucleotides or non-phosphodiester bonds, for example, that are designed to increase biological stability of the inhibitory nucleic acid or to increase intracellular stability of the duplex formed between the inhibitory nucleic acid and the target nucleic acids.

An inhibitory nucleic acid may be prepared using available methods, for example, by expression from an expression vector encoding a complementarity sequence of the nucleic acids described herein. Alternatively, it may be prepared by chemical synthesis using naturally occurring nucleotides, modified nucleotides or any mixture of combination thereof. In some embodiments, the nucleic acids described herein are made from modified nucleotides or non-phosphodiester bonds, for example, that are designed to increase biological stability of the nucleic acids or to increase intracellular stability of the duplex formed between the inhibitory nucleic acids and other (e.g., endogenous) nucleic acids.

For example, nucleic acids can be peptide nucleic acids that have peptide bonds rather than phosphodiester bonds.

Naturally occurring nucleotides that can be employed in the nucleic acids include the ribose or deoxyribose nucleotides adenosine, guanine, cytosine, thymine and uracil. Examples of modified nucleotides that can be employed in the nucleic acids include 5-fluorouracil, 5-bromouracil, 5-chlorouracil, 5-iodouracil, hypoxanthine, xanthine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methythio-N6-isopentenyladeninje, uracil-5oxyacetic acid, wybutoxosine, pseudouracil, queosine, 2-thiocytosine, 5-methyl-2-thiouracil, 2-thiouracil, 4-thiouracil, 5-methyluracil, uracil-5-oxacetic acid methylester, uracil-5-oxacetic acid, 5-methyl-2-thiouracil, 3-(3-amino-3-N-2-carboxypropyl) uracil, (acp3) w, and 2,6-diaminopurine.

Thus, inhibitory nucleic acids described herein may include modified nucleotides, as well as natural nucleotides such as combinations of ribose and deoxyribose nucleotides. The inhibitory nucleic acids and may be of same length as wild type. The inhibitory nucleic acids described herein can also be longer and include other useful sequences. In some embodiments, the inhibitory nucleic acids described herein are somewhat shorter. For example, inhibitory nucleic acids described herein can include a segment that has a nucleic acid sequence that can be missing up to 5 nucleotides, or missing up to 10 nucleotides, or missing up to 20 nucleotides, or missing up to 30 nucleotides, or missing up to 50 nucleotides, or missing up to 100 nucleotides from the 5′ or 3′ end.

According to the methods of the present invention, the method can be used therapeutically (e.g., in order to treat an individual that has been infected with the virus or has developed the cancer that is being targeted), or prophylactically (e.g., in order to protect an individual against becoming infected with the virus or developing the cancer that is being targeted).

### Methods for Preparing Polymer Dot Bioconjugates

As described herein, the polymer dots provided by the present invention allow for the conjugation of biological molecules to Pdots that can be used in a wide array of diagnostic, therapeutic and experimental assays. In this fashion, a molecule, such as a biomolecule, may be attached to a polymer dot through adsorption to the surface of the polymer dot (e.g., mediated through electrostatic or hydrophobic interactions) or by direct chemical attachment.

As used herein, a “bioconjugated Pdot” refers to a polymer dot with any biomolecule stably attached through any stable physical or chemical association.

### 1. Bioconjugation Through Physical Adsorption

In one embodiment, biomolecule is stably associated with the polymer dots by physical adsorption. Physical adsorption can arise from a range of forces, including but not limited to van de Waals, electrostatic, pi-stacking, hydrophobic, entropic forces and combinations thereof. Physical adsorption will be mediated by the physical and chemical properties of the polymer dot and the target molecule (e.g., biomolecule) being adsorbed. In one embodiment, the Pdots were synthesized using DSPE-PEG-PEI as an amphiphilic crosslinker and were positively charged in aqueous solution. It is believed that that siRNA, which is negatively charged, will bind to positively charged Pdots.

### 2. Bioconjugation Through Chemical Bonding

In one embodiment, a molecule (e.g., a biomolecule) is attached to the polymer dot by chemical bonding, which uses functional groups be available on the polymer dot, such as carboxylic acid, amino, mercapto, azido, alkyne, aldehyde, hydroxyl, carbonyl, sulfate, sulfonate, phosphate, cyanate, succinimidyl ester, substituted derivatives thereof, or combinations thereof. In general, any functional groups that allow bioconjugation may be used. Such groups could be found by one of ordinary skill in the art, for example in Bioconjugate Techniques (Academic Press, New York, 1996 or second edition, 2008; the disclosures of which are herein incorporated by reference in their entireties for all purposes). Then the bioconjugation can be done by standard bioconjugation techniques.

### Methods for Imaging and Molecular Labeling

The use of fluorescent polymers for in vivo imaging and molecular labeling has several advantages over the materials currently in use. For example, fluorescent polymer dots possess high fluorescence brightness/volume ratios, have high absorption cross sections, high radiative rates, high effective chromophore density, and minimal levels of aggregation-induced fluorescence quenching. The use of fluorescent polymer dots as fluorescent probes also confers other useful advantages, such as the lack of heavy metal ions that could leach out into solution.

### EXAMPLES

The disclosure can be better understood by reference to the following examples which are offered by way of illustration. The disclosure is not limited to the examples given herein.

### Example 1

### Introduction

Historically, the three major treatment paradigms for cancer include surgery, chemotherapy, and radiotherapy. In the last decade, gene/immune therapy became the fourth avenue for cancer therapeutic strategies (1). Compared to the three traditional treatment approaches, gene/immune therapy has shown fewer side effects and can provide higher cancer target efficiency (2,3). Other approaches have used anti-angiogenesis therapies in combination with chemo- and radiotherapy treatments, with mixed beneficial outcomes (4). More recent efforts have focused on development of gene silencing pathways for which RNA interference has emerged as a tool for transcriptional/translational silencing of target genes (5,6). Short interfering RNA (siRNA) is a short RNA of 20-25 base pairs (bp), which can directly disrupt targeted mRNAs and lead to downregulation of target gene expression (7). Moreover, siRNA is negatively charged at physiological pH and can be constructed using exterior cationic materials as a safe siRNA delivery system for biological applications from basic research to clinical use (8,9).

One of the main challenges for siRNA gene therapy lies in the siRNA delivery process, itself. Factors contributing to inefficient delivery include poor stability of siRNA in circulation (10), siRNA degradation by nucleolytic enzymes (6), and siRNA destruction by self-immune recognition (6). Therefore, improving the delivery efficiency of siRNA has become a factor for the development of siRNA-based gene therapy approaches. Suitable delivery platforms might provide protection for the siRNA, thus improving gene delivery efficiency. Traditionally, liposomes as well as inorganic and polymer materials have served as biomolecule-delivery platforms to control payload release, for example, in the context of drug encapsulation and release (11). Li and colleagues constructed aromatized liposomes for systemic delivery, anticipated to extend duration of release and slow loss of particles from the site of injection (11). Liposomes have a phospholipid bilayer structure to form spheres for drug loading to ensure a long circulation time; however, in vivo stability is a challenge for liposomes due to toxicity and poor local accumulation (12,13). Inorganic nanoparticles, such as carbon nanotubes, iron oxide nanoparticles, and mesoporous silica nanoparticles, have been utilized for drug delivery in a variety of systems (14,15). Although these inorganic nanoplatforms have shown good stability as delivery vehicles for anticancer drugs or siRNA, controllable drug or siRNA release and visual monitoring are often problematic.

To address these challenges, a polymer-based matrix has been developed to form polymer dots (Pdots) as a delivery platform for siRNA (Pdot-siRNA). Due to the advantages of Pdots, such as controlled release, protection, and specific targeting ability of biomolecules, Pdots are an ideal nanocarrier for therapeutic agents such as conventional drug molecules or nucleic acid delivery (16,17). Pdots have been developed for a wide variety of biomedical applications. One example is in photodynamic therapy (PDT) for cancer cell treatment using the murine breast cancer cell line, 4T1 cells, that were investigated for in vitro PDT (18) and photothermal therapy (PTT) (19). The leading rationale was to leverage the advantages of siRNA-based inhibition of gene expression to engineer a nanomaterials-based platform to target specific genes. As proof-of-concept, the Pdot-siRNA approach offers specific inhibition of gene expression in combination with delivery visualization. This approach has been successfully tested in a primary culture of metabolically active brain microvascular-derived endothelial cells.

### Materials and Methods

### Selection of Polymers

The dual functional tool was made using three polymers. The first is hydrophobic Poly[2-methoxy-5-(2-ethylhexyloxy)-1,4-phenylenevinylene] (MEHPPV), that is a semiconducting polymer with low molecular weight and hydrophobic characteristics. The second is Poly[2,6-(4,4-bis-(2-ethylhexyl)-4H-cyclopenta[2,1-b;3,4-b′]dithiophene)-alt-4,7 (2,1,3-benzothiadiazole)] (PCPDTBD), that is a semi-conducting polymer. The third is a positively charged amphiphilic polymer 1,2-distearoyl-sn-glycero-3-phosphoethanolamine-polyethylene glycol-polyetherimide (DSPE-PEG-PEI) that is synthesized using 1,2-distearoyl-sn-glycero-3-phosphoethanolamine-N-(succinimidyl (polyethylene glycol)-3400) (DSPE-PEG-NHS) and polyethylenimine (PEI, MW 25,000). The latter is a poly(phenylenevinylene) (PPV) derivative and a conjugating polymer with its highest occupied molecular orbital (HOMO) below the fermi level of gold. PCPDTBT is also a semiconducting polymer used to form a donor/acceptor (D-A) blend with a variety of conducting polymers which can be used to enhance the power conversion efficiency (PCE) in an electrochemical device (20), however herein it is used in a cell culture system. MEHPPV can be blended with PCPDTBT for use as a sensitizer for dye sensitized photo sensors (DSPS). These D-A conjugated polymers with NIR absorbance peak have been used to achieve efficient photothermal therapy (PTT) (21) in other systems.

### Materials and Instruments

The polymers MEHPPV, PCPDTBT, PEI, organic solvent tetrahydrofuran (THF), 4-(2-Hydroxyethyl) piperazine-1-ethanesulfonic acid, N-(2-Hydroxyethyl) piperazine-N′-(2-ethanesulfonic acid) (HEPES), dimethyl sulfoxide (DMSO), Amicon® Ultra-4 centrifuge filters with regenerated cellulose 100 K molecular weight cut-off (MWCO) and penicillin-streptomycin were purchased from Sigma Aldrich (St. Louis, MO, USA). DSPE-PEG-NHS, MW 3400 was purchased from NANOCS (New York, NY, USA). Deionized (DI) water (18 mΩ cm at 25° C.) was used in the experiments. Primary cultures of brain microvascular fibroblast cells (BMVFb) and brain microvascular endothelial cells (BMVEC) derived from CD1 adult mouse brain were purchased from Cell Biologics (Chicago, IL, USA). Dulbecco's modified Eagle medium with Glutamax (DMEM), Opti-MEM® Medium, Fetal Calf Serum, Penicillin-Streptomycin (10,000 IU Pen; 10,000 μg Strep in a 100× stock solution), Electron Microscopy Sciences (EMS) 16% Paraformaldehyde (PFA) aqueous solution, Phosphate buffered saline (PBS, 1×), cell culture plates, 8-well chambered cover glass w/non-removable wells, 96-well plates, 4′,6-diamidino-2-phenylindole (DAPI) (cat #83210, 5 μg/mL), Alexa Fluor 488˜phalloidin (cat #A12379, 1U in methanol), the Glyceraldehyde phosphate dehydrogenase (Gapdh) antibody (cat #MA1-16757, 1:100, 10 μg/mL), the donkey anti-rabbit IgG conjugated to Cy3 (cat #711-166-152, 7.5 μg/mL), the Live/Dead Cell Imaging Kit, Invitrogen CyQUANT™ Lactate Dehydrogenase (LDH) Cytotoxicity Assay kit and Lipofectamine™ RNAiMAX Transfection Reagent were purchased from ThermoFisher Scientific (Waltham, MA, USA). The Maxwell® RSC simplyRNA extraction kit for cells was purchased from Promega (Madison, WI, USA). The immunolabeling block solution was prepared in the lab as previously described (22,23). The Vectashield hardset mounting medium was purchased from Vector Laboratories (Burlingame, CA, USA). Lab-Tek II 8-well Chamber Slides were purchased from Nalgene-Nunc International Corp (Naperville, IL, USA). Micro coverglass coverslips were purchased from Sargent-Welch VWR Scientific (Buffalo Grove, IL, USA). The cell counting system used was a LUNA-II Cell Counter from Logos (Biosystems, Annandale, VA).

A Hitachi 7500 transmission electron microscope (Hitachi, Tokyo, Japan) was used to observe the morphology of Pdots and Pdot-siRNA. At 25° C., the particle size and zeta potential of Pdots and Pdot-siRNA was determined using Zetasizer Nano-ZS dynamic light scattering (DLS) (Malvern Panalytical, UK) with a 633 nm Helium-Neon laser and backscattering detection. The fluorescence spectra and photostability measurements were obtained using a Shimadzu RF-6000 spectrophotometer (Shimadzu, Tokyo, Japan). UV-visible absorption studies were performed using a PerkinElmer Lambda 1050 UV-Vis-NIR spectrophotometer (Akron, OH, USA). An Olympus IX50/IX70 inverted system microscope (Olympus, Center Valley, PA) and captured using SimplePCI (v. 6.1) software. An Olympus FV3000 laser scanning confocal microscope (Olympus, Center Valley, PA) was used for the in vitro cell immunolabel imaging of Pdots and Pdot-siRNA. Benchmark Multi-Therm™ shaker (Sigma Aldrich, St. Louis, USA) was used to conjugate DSPE-PEG-NHS with PEI. Branson sonicator 3800 (Emerson, St. Louis, USA) was used to help to synthesis of Pdots in the solution. The polymers' 1H-NMR spectra were detected using an AVANCE III spectrometer (HD 400 MHZ, Bruker, Switzerland). NuAire Biosafety level II sterile hood (model NU.425.400, NuAire, Plymouth, MN) was utilized for all cell culture experiments.

### Synthesis of Dual-Emissive Pdots

Synthesis of positively charged DSPE-PEG-PEI amphiphilic polymer was based on the reaction via primary amine functional groups from PEI and the NHS ester moiety from DESP-PEG-NHS. Briefly, 0.8 mL of 10 mg/mL in DMSO was prepared in a 1.5 mL centrifuge tube, and then functionalized with 0.2 mL of 5 mg/mL DSPE-PEG-NHS in DMSO added into the above solution under shaking conditions for 24 h at 1000 rpm at room temperature. The resulting mixture was concentrated and purified using Amicon® Ultra-4 regenerated cellulose centrifugal filter with 100 K MWCO. The chemical structure of DSPE-PEG-PEI amphiphilic polymer was confirmed by ¹H NMR (in D2O).

The dual-emissive Pdots were synthesized by using a nanoprecipitation method (24) with a minor modification. Briefly, 1.0 mg/mL of MEHPPV, 1.0 mg/mL of PCPDTBT were mixed in tetrahydrofluorane (THF) and 1.0 mg/mL of DSPE-PEG-PEI in DMSO were prepared as a working solution with 10.0 mL of DI water in a glass vial. Then, 1.0 mL of 1.0 mg/mL MEHPPV, 0.5 mL of 1.0 mg/mL PCPDTBT, and 0.1 mL of 1.0 mg/mL DSPE-PEG-PEI were mixed well with rapid, repeated pipetting in 3.4 mL of THF, with the total volume set at 5.0 mL. The mixture was quickly injected into 10.0 mL of DI water under simultaneous ultrasonication (50-60 Hz) for 2 min. The THF solvent was evaporated off in the fume hood at room temperature overnight after which the Pdots solution in water was purified using a 0.22 μm VWR® syringe filter (VWR), following by centrifugation and concentration using Amicon® Ultra-4 regenerated cellulose centrifugal filter with a 100 K MWCO. The glass vial containing the final product in the 20 mM HEPES buffer was wrapped in tin foil to protect the nanomaterials from light, prior to storage at 4° C.

Electrostatic Binding of siRNA with Pdots (Pdot-siRNA)

Based on their respective charges, the Pdots and siRNA are likely bound via an electrostatic interaction. The Pdots were synthesized using DSPE-PEG-PEI as an amphiphilic crosslinker and were positively charged in aqueous solution. siRNA, which is negatively charged, binds to positively charged Pdots. To generate the Pdot-siRNA, commercially available siRNA with two different sequences-control siRNA and Gapdh-specific siRNA was utilized. The rationale for this approach was to include a specific target siRNA (Gapdh) and a scrambled sequence control siRNA for comparison. To generate the Pdot-siRNA, 5.0 μg/mL Pdots were combined with different amounts of siRNA (2 nmole, 1 nmole, 0.5 nmole, 0.25 nmole, 0.125 nmole and 0.0625 nmole) in HEPES buffer in centrifuge tubes under shaking conditions for 30 min at 4° C. at 200 rpm followed by incubation at 4° C. for 30 min. The final Pdot-siRNA was purified and centrifuged using the 100 K MWCO Amicon® Ultra-4 centrifuge filter. The characterization of the Pdots bound to the siRNA was investigated by UV-Vis absorption.

### Cell Culture

Primary cultures of mouse-brain-derived microvascular fibroblast cells (BMVFb) and endothelial cells (BMVEC) were obtained from CD1 adult mouse brain and maintained in complete DMEM with 1× Glutamax and 4.50 g/L glucose supplemented with 10% fetal bovine serum (FBS), penicillin solution (100 U/mL), and streptomycin (100 μg/mL). The pellet was resuspended into 7.5 mL of complete DMEM with 15% FBS and 20 μg/mL Fibroblast growth factor 2 (Fgf2) along with penicillin-streptomycin 100 μg/mL stock solution. The cell lines were cultured at 37° C. under a humidified atmosphere containing 5% CO2. Cell counts for assay plating were determined by counting a diluted suspension with a LUNA-II automated cell counter.

Efficacy Test of siRNA

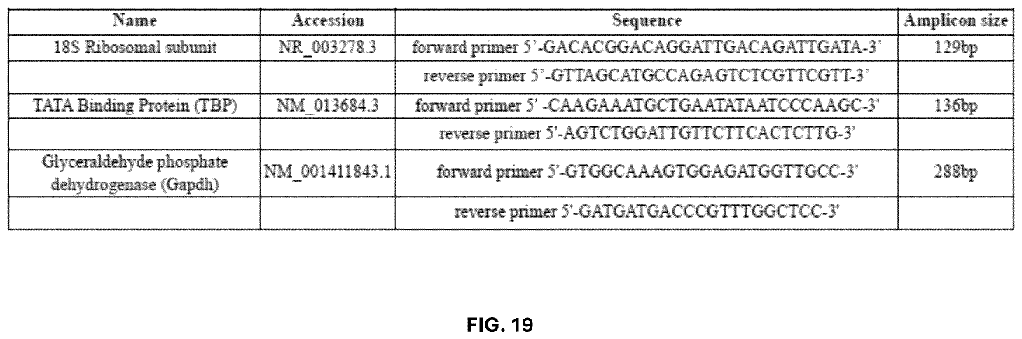

Commercially available control siRNA sequences (Cat. #4390844 and 4390847, sequence proprietary) and Gapdh siRNA sequences (Cat. #4390850, sequence proprietary) were obtained in lyophilized powder form and were resuspended with nuclease-free water to a stock concentration of 100 μM and stored at −20° C. To establish baseline efficacy for gene expression inhibition, BMVEC were seeded in a 24-well culture plate at ˜50,000 cells per well and cultured for 48 h. The medium was removed, and the cells were incubated in 0, 5, 10, and 20 μM control or Gapdh siRNA with Lipofectamine® RNAiMAX Reagent as the delivery mechanism in Opti-MEM® Medium following the manufacturer's recommended protocol. After 4 h, the transfection mixture was removed and replaced with standard DMEM culture medium. After 24 h from the initial point of siRNA treatment, the medium was removed, the cells were washed once with 1×PBS on ice and the total RNA extracted using the Maxwell® RSC simplyRNA extraction kit for cells (Promega, Cat. #AS1390) following manufacturer's recommended protocol. In brief, cells were lysed in 200 μl of Thioglycerol and Homogenization Solution followed by treatment with DNase I Solution to eliminate contaminating genomic DNA. After RNA purification, the RNA Integrity Number (RIN), as a reflection of RNA quality, and the concentration of the total RNA were determined using the High Sensitivity RNA ScreenTape system (Agilent Technology, 2200 TapeStation) and only samples with RIN>9.0 were used for generating complementary DNA (cDNA). In order to quantify changes in gene expression across samples, cDNA synthesis and quantitative PCR (qPCR) were conducted as described (23). In brief, 200 ng of total RNA was mixed to a final concentration with 2.5 U/μL of Murine Moloney Leukemia Virus Reverse Transcriptase (MMLV-RT), 1.25 mM nucleotides, 0.5 mM oligo dT, 2.5 mM random Hexamers, 1 U/μL RNase Inhibitor, and 1× Reverse Transcriptase buffer and incubated for 1 h at 42° C. to generate the cDNA library. Library quality was confirmed based on uniform sample expression of the 18S Ribosomal subunit as a reference gene with primers as described in supporting materials (FIG. 19).

### Pdots Cellular Uptake, Signal Persistence, and Determination of In Vitro Cytotoxicity

To assess cellular uptake ability and determine toxicity, the effect of Pdots was evaluated at different concentrations in primary cultures of BMVFb cells. The BMVFb were cultured in 8-well Lab Tek Chamber slides at a target plating density of 2,500 cells/well. Four different incubation times (4 h, 8 h, 12 h, 24 h) were assessed with four different Pdots concentrations (20 μg/mL, 10 μg/mL, 5 μg/mL and control) in standard culture medium using four technical replicates for each time points and conditions. In brief, the cells were washed, fixed and blocked at each incubation time and concentration. The labelling mix included Alexa Fluor 488˜phalloidin (1:300) and DAPI (1:1000) which was added to each slide. The slides were washed with 1×PBS and ProLong anti-fade mountant medium was added to each slide prior to coverslipping.

The cells were visualized using an Olympus FV3000 laser scanning confocal microscope under 20× objective and images were collected in each channel were postprocessed to generate composites using Adobe Photoshop. The DAPI signal (blue, detection wavelength 430-480 nm), the Alexa Fluor 488 Phalloidin signal (green, 500-550 nm), and the Pdots (red, 550-650 nm) were collected from three independent images with 4 regions collected for each image. Cell viability under various incubation times and concentrations was summarized. Live and dead cells could be recognized based on the shape of nuclei. These images were analyzed with NIH Image J (25) to quantify the number of live cells (large, round nuclei) versus dead cells (small, pyknotic nuclei). Determination of cellular uptake for Pdot-siRNA and immunolabeling

The BMVEC were cultured for 24 h in 8-well Lab Tek Chamber slides at a density of 50,000 cells/well to allow the cells to adhere fully to the plastic. The media was removed and replaced with 300 μL/well of freshly prepared Pdot-control siRNA at different amounts of siRNA ranging from 0-2 nmole with an additional control well of 5 μg/mL of Pdots only. The cells were incubated with the Pdot-control siRNA for 4 h to allow cellular uptake after which the media was removed, and the cells were fixed as described above. To visualize the cellular architecture, the cells were immunolabelled with block solution containing Alexa Fluor 488 phalloidin and DAPI. The control and treated cells were imaged using the Olympus FV3000 Laser Scanning Confocal Microscope (3 times line average, line sequential scan, auto confocal aperture) and the objective lens was UPLFLN 40× oil with NA 1.3.

### Investigation of In Vitro GAPDH Knockdown

To determine if the Pdot-siRNA could be used to inhibit Gapdh expression, the BMVEC were cultured in a 24-well plate at a density of 50,000 cells/well and then treated with either Pdot-siRNA control or Pdot-siRNA Gapdh for comparison. A comparison of Pdot alone (5 μg/mL), control siRNA alone (1 nmole), Pdot-control siRNA (0.5 and 1.0 nmole), and Pdot-Gapdh siRNA (0.5 and 1.0 nmole) was generated with four replicates for each condition. The cells were treated in standard culture medium for 4 h, after which the treatment medium was removed, and the cells were culture for an additional 20 h in normal growth media (DMEM with 10% FBS). The cells were collected afterward to proceed with total RNA isolation, cDNA synthesis, and quantitative real-time polymerase chain reaction (qPCR).

The relative concentration of Gapdh transcript from BMVEC treated with Pdot-Gapdh siRNA was determined using qPCR. Primers specific to Gapdh and TATA binding protein (TBP) were designed to cross intron-exon borders and the 288 bp fragment (Gapdh) and 136 bp fragment (TBP) were sequenced to confirm target specificity. Total RNA (400 ng) from treated cells was converted to cDNA as previously described. The qPCR amplification mixes included 2 μL of cDNA, 5 μL of 2× Absolute Blue qPCR SYBR Green with ROX (ThermoFisher Scientific, Waltham, MA), and 1 uL each of forward and reverse primers (10 μM). The cycle profile was 95° C. for 1 min followed by 30 cycles alternating 60° C. with 72° C. extension using the StepOnePlus™ Real-Time PCR Systems (Applied Biosystems). The modified delta-delta C_{T} (ΔΔC_{T}) method was used to calculate the Gapdh to TBP ratio for each sample (26). This approach enhances the precision of each sample's relative gene expression quantitation by using the geometric mean of the sample set for comparison. The difference in C_{T} values (ΔC_{T}) of the targeted gene was determined by subtracting the C_{T} of the target gene of each sample from the C_{T} of the Reference gene. The ΔC_{T} of the reference gene was obtained by subtracting the geometric mean of the target gene C_{T} from the geometric mean of the reference gene C_{T}. This was used to normalize the relative change in expression for each sample (ΔΔC_{T}) by subtracting the ΔC_{T} of the target gene from the ΔC_{T} of the reference gene. The fold change in gene expression for each sample was determined using log 2 ΔΔC_{T} and the ratio graphed as mean and standard deviation from the mean.

### Gapdh Immunolabeling in BMVEC

After Pdot-siRNA treatment, cells were fixed in 4% PFA in PBS for 15 min at RT followed by additional PBS washes. Cells were blocked and permeabilized with 3% donkey serum, 1% bovine serum albumin (BSA), 0.1% saponin, 0.1% Triton X-100, and PBS for 30 min at RT. Cells were labelled with Gapdh antibody for 90 min at RT followed by three 10 min washes with PBS. Primary antibody was bound with donkey anti-rabbit IgG˜Cy3 for 60 min at RT followed by three 10-min washes with PBS. Slides were mounted with ProLong anti-fade and a glass coverslip prior to confocal imaging as described above.

### Results and Discussion

### Preparation of Positively Charged DSPE-PEG-PEI Amphiphilic Polymer

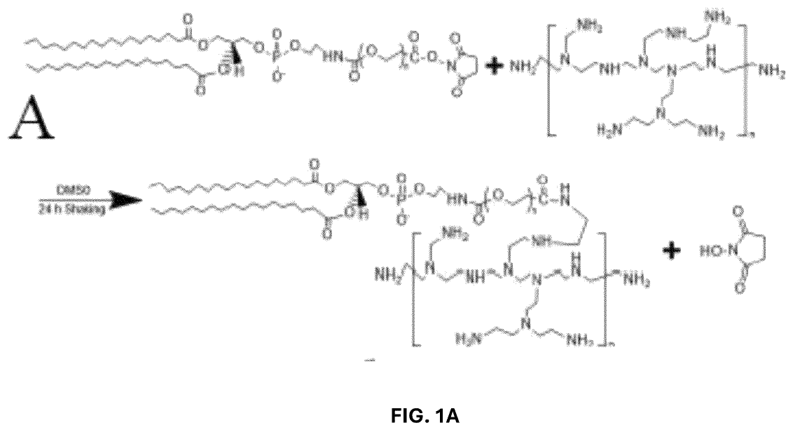

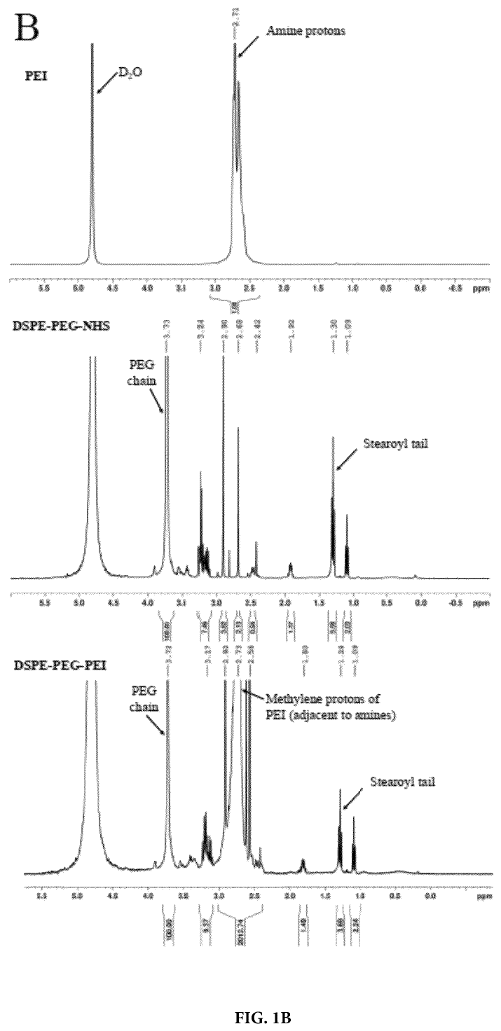

The phosphoethanolamine-polyethylene glycol polymers (PE-PEG) based nanoplatform is a promising nanoparticle delivery system because of its significant advantages, including biocompatibility, prolonged circulation, bioaccumulation in tumor cells and ability to readily incorporate drugs such as doxorubicin (DOX) to increase cancer cell internalization and enhance cytotoxicity (27-29). DSPE-PEG-NHS provided an N-hydroxysuccinimide (NHS) ester for conjugation with amine-containing molecules/chemicals which was used to conjugate with PEI via the primary amine-reactive NHS ester moiety. The structure of DSPE-PEG-NHS, PEI, and the resulting DSPE-PEG-PEI copolymer were verified by ¹H NMR (FIG. 1). The deuterium oxide (D2O) peak was detected at 4.8 ppm. The peaks of DSPE (1.0-1.5 ppm, stearoyl), PEG (3.7 ppm, —CH₂O—), NHS (2.7 ppm, —NCOCH₂CH₂—) and PEI (2.5-3.0 ppm, —NHCH₂CH₂NH2) were confirmed using NMR spectroscopy. The ¹H-NMR spectrum of DSPE-PEG-PEI in D20 exhibited characteristic peaks at 2.5-3.0 ppm were from methylene protons of PEI which was adjacent to amine functional groups, indicating that PEI was successfully introduced to the DSPE-PEG-NHS molecule.

Rational Design of Pdots for siRNA Delivery

To develop a stable and dual-functional Pdots nanoplatform for siRNA delivery, three types of polymers were selected for the synthesis of Pdots, including hydrophobic Poly[2-methoxy-5-(2-ethylhexyloxy)-1,4-phenylenevinylene] (MEHPPV) and Poly[2,6-(4,4-bis-(2-ethylhexyl)-4H-cyclopenta[2,1-b;3,4-b′]dithiophene)-alt-4,7 (2,1,3-benzothiadiazole)] (PCPDTBD), positively charged amphiphilic polymer 1,2-distearoyl-sn-glycero-3-phosphoethanolamine-polyethylene glycol-polyetherimide (DSPE-PEG-PEI). MEHPPV and PCPDTBT are semiconducting polymers with strong x-electrons, contributing to a donor-acceptor type conjugation system responsible for the absorption and emission of optical properties (30-32). The DSPE-PEG-PEI was synthesized using 1,2-distearoyl-sn-glycero-3-phosphoethanolamine-N-[succinimidyl (polyethylene glycol)-3400] (DSPE-PEG-NHS) and polyethylenimine (PEI, MW 25,000), and NHS ester can be easily incorporated into liposome and other chemicals via the reaction of NHS and amine groups. DSPE-PEG-PEI can both provide a positively charged surface on the Pdots and act as backbones in the Pdots to support stable and crosslinked nanoparticle formation.

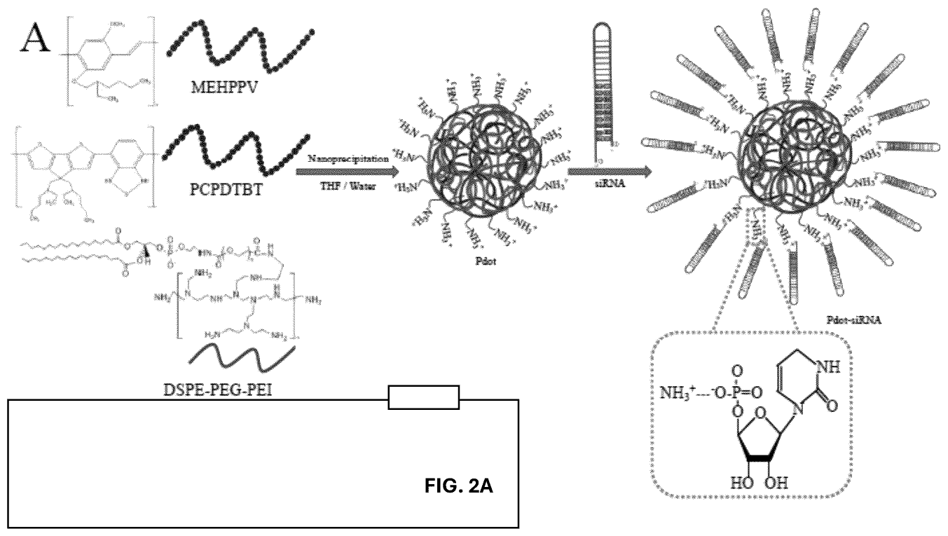

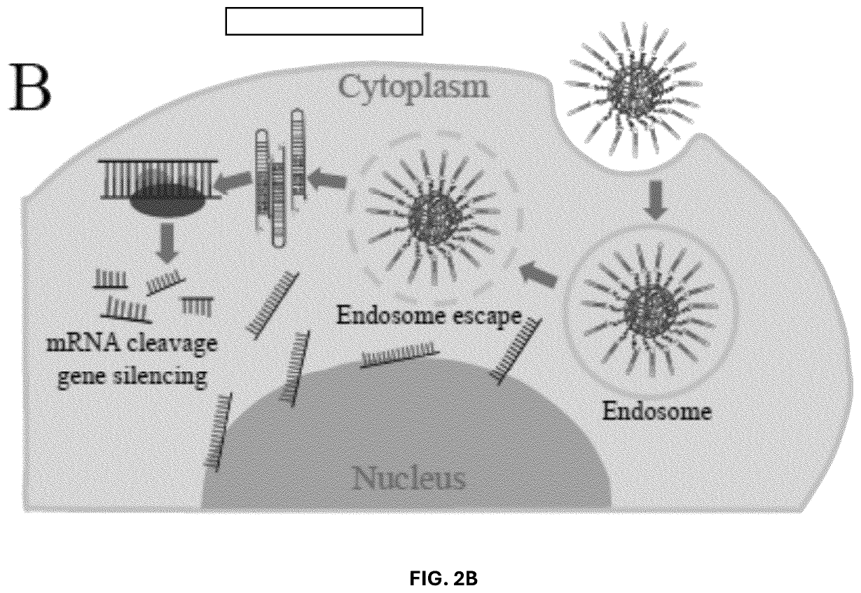

The synthesis approach to generate Pdots is based on the nanoprecipitation method as shown in the schematic (FIG. 2A). In brief, 1.0 mL of 1.0 mg/mL MEHPPV and 0.5 mL of 1.0 mg/mL PCPDTBT were mixed in a water-miscible organic, solvent-tetrahydrofuran (THF). 0.1 mL of 1.0 mg/mL amphiphilic polymer DSPE-PEG-PEI in DMSO was added to the polymer mixture above. A total of 5.0 mL of these polymer solutions were mixed well and rapidly added to an excess of 10.0 mL DI water under 50-60 Hz ultrasonic dispersion conditions for 2 min. Due to the presence of the amphiphilic polymer DSPE-PEG-PEI, Pdots have amine functional groups on the surface and can be dispersed in the aqueous solution. The current model of how the Pdot-siRNA is taken up and works in cells is as follows. The 3′ phosphate group on the ribonucleotide strand confers a negative charge; therefore, the siRNA electrostatically binds to the outside surface of the Pdot nanoplatform that is positively charged (FIG. 2B). When the Pdot-siRNA nanoplatform is taken up by the cells, the nanoplatform may be encapsulated within an endosome structure and delivered to the perinuclear region of the cell via a mechanism that has been described previously in the literature (33). Once the Pdot-siRNA has escaped the endosome compartment, the siRNA will be released from Pdots platform and bind to its mRNA target via base-pair complementarity. Binding of the siRNA to its specific target will prevent translation by the ribosomal complex or target the double-stranded RNA for degradation, resulting in reduced protein product for that target transcript. This approach allows for the dual advantage of being able to deliver siRNA specific to critical cellular targets and modify gene expression while simultaneously imaging exactly where the siRNA has been delivered. This tool has broad use in basic and clinical research, as well as therapeutic use.

### Characterization of Pdots and Pdot-siRNA

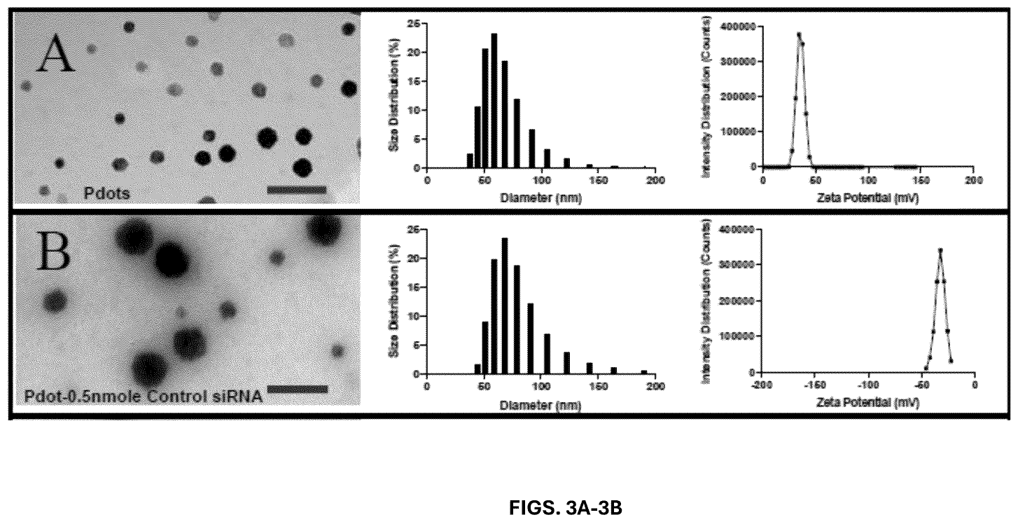

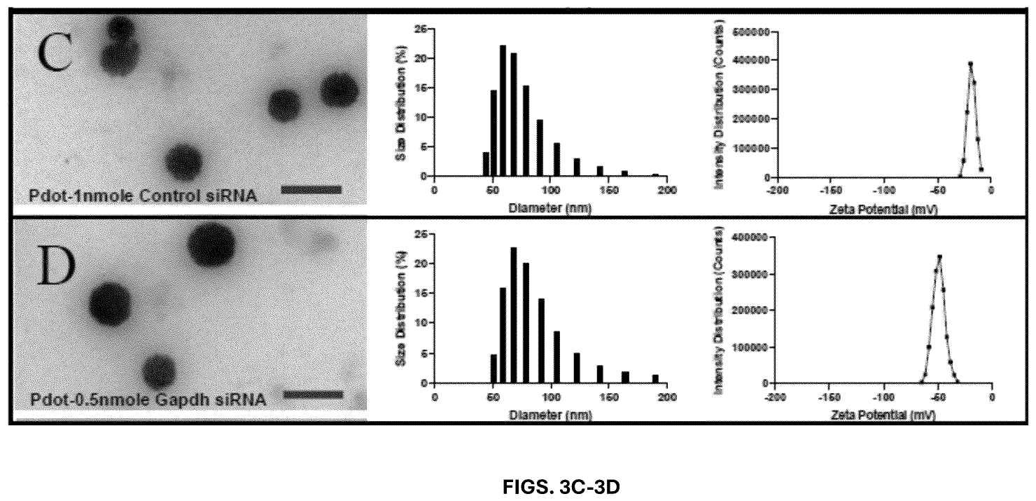

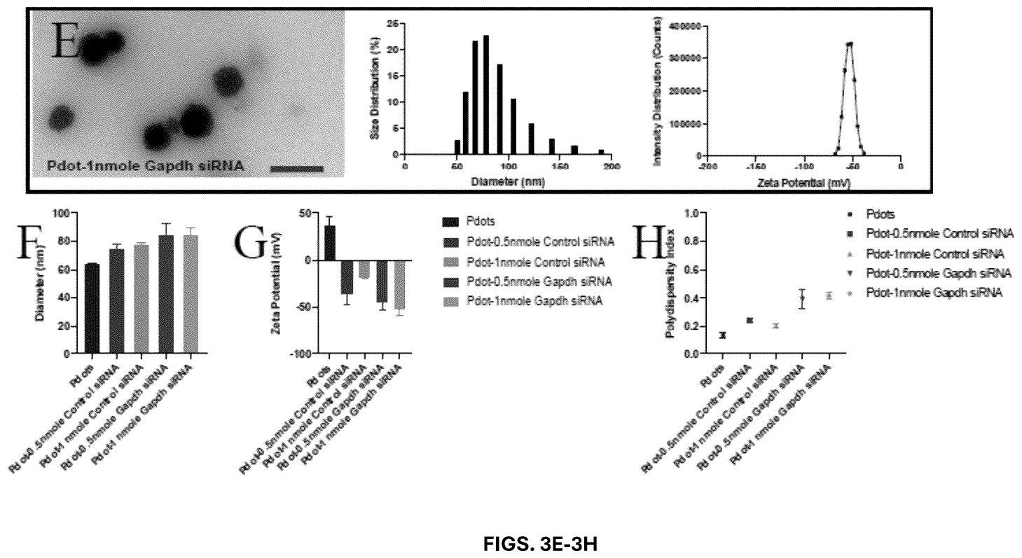

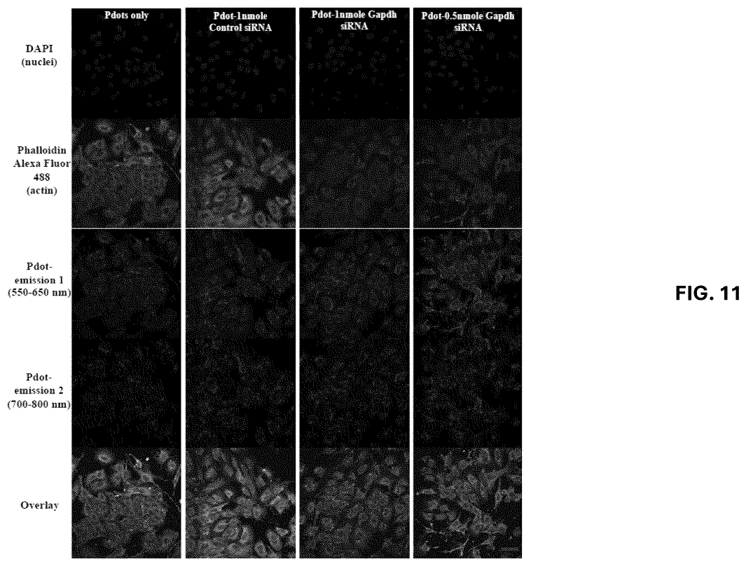

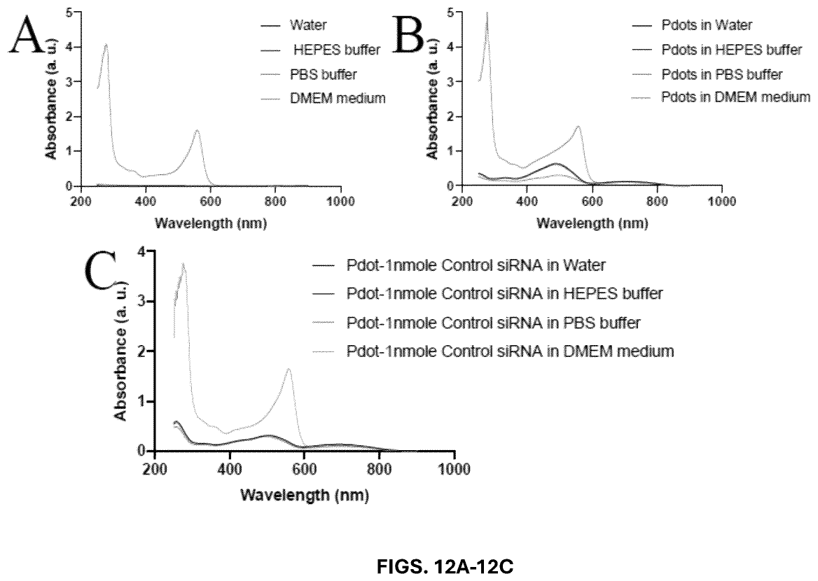

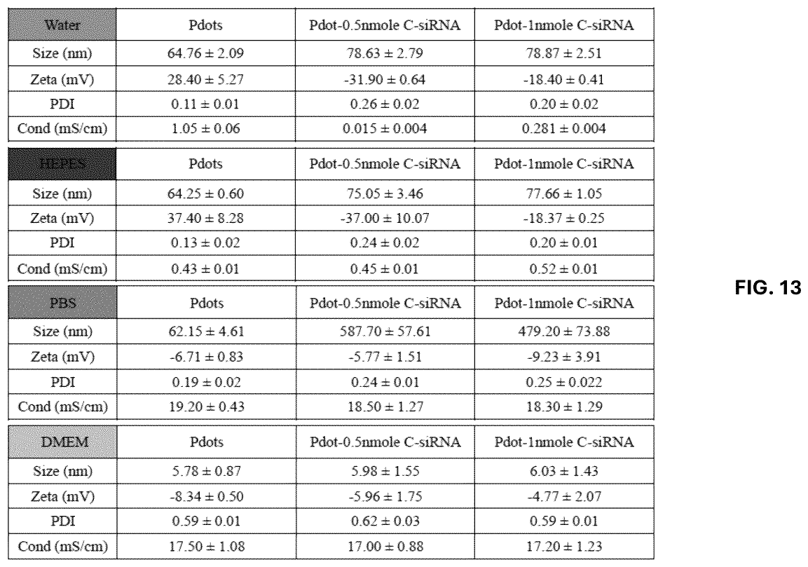

The morphology and surface charge of Pdots, Pdot-control siRNA, and Pdot-Gapdh siRNA were characterized using TEM and DLS (FIG. 3) based on the nanomaterials dispersed in HEPES buffer. The TEM images showed that Pdots, Pdot-control siRNA, and Pdot-Gapdh siRNA were successfully synthesized and dispersed in the solution without aggregation. The hydrodynamic diameter of Pdots was measured to be 64.25±0.60 nm with positively charged zeta potential (+37.40±8.28 mV) around the surface in HEPES buffer (FIG. 3A). The diameter of Pdots with 0.5 nmole of control siRNA and Pdots with 1.0 nmole of control siRNA were measured to be 75.05±3.46 nm and 77.66±1.05 nm, with the zeta potential of each being negatively charged at −37.00±10.07 mV and −18.37±0.25 mV, respectively (FIG. 3B, 3C). In comparison, the diameter of Pdots with 0.5 nmole of Gapdh siRNA and Pdots with 1.0 nmole of Gapdh siRNA was measured to be 84.50±8.45 nm and 82.27±9.83 nm, with the zeta potential values determined to be −45.77±7.16 and −52.00±6.05 mV, respectively (FIG. 3D, 3E). The average size comparison between Pdots and different amounts of Pdots bound to control or Gapdh siRNA was shown in FIG. 3F. As expected, Pdots are positively charged; however, when Pdots bind with control or Gapdh siRNA the overall zeta potential is negative (FIG. 3G). The polydispersity index (PDI) confirmed that Pdots have a narrow particle size distribution in HEPES buffer, and Pdots with siRNA showed a relatively high particle size distribution. During the characterization of the Pdots and Pdot-siRNA, absorbance, size, and zeta potential were measured in water, biocompatible buffer (HEPES and PBS), and cell culture medium (DMEM). This latter series of control tests revealed that the nanomaterial average diameter differed in buffer versus cell culture media (FIGS. 11 & 12).

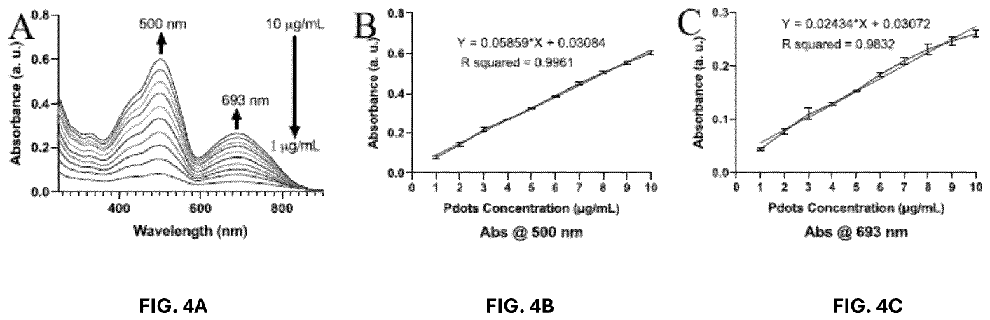

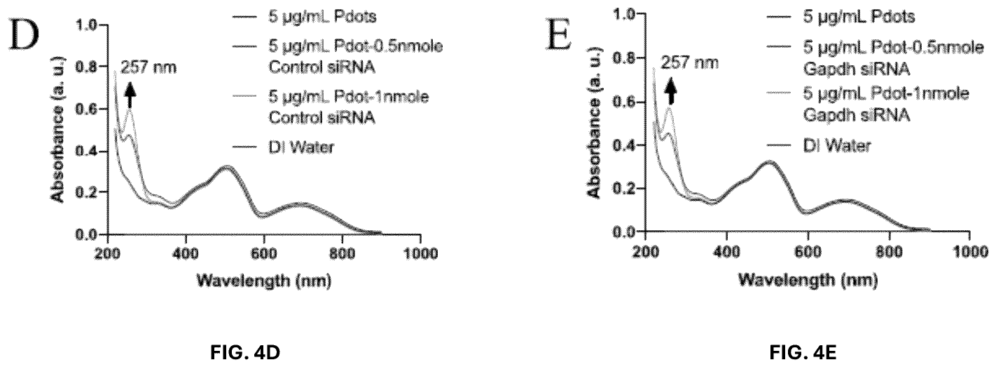

In addition to the delivery function of the Pdots nanoplatform, there is also an advantage from the real-time monitoring and detection via fluorescence emission of the Pdots. In order to determine the Pdots' absorbance and emission properties, a concentration series from 1 μg/mL to 10 μg/mL was analyzed using a UV/Vis/NIR spectrometer (FIG. 4A). The absorbance spectra showed two absorbance peaks at 500 nm and 693 nm, respectively. Linear regression analysis predicted the Pdots' concentration based on absorbance value. A linear relationship for both 500 nm and 693 nm, relative to concentration, with R square equal to 0.9961 and 0.9832 (FIGS. 4B and 4C), respectively, indicates a tight correlation between these two parameters. Once the siRNA was associated with the Pdots, an additional peak at 257 nm was observed, which is characteristic of nucleotide absorbance properties. These results indicate that siRNA bound to Pdots shifts the absorbance properties to include that associated with the nucleotides for both control siRNA (FIG. 4D) and Gapdh siRNA (FIG. 4E).

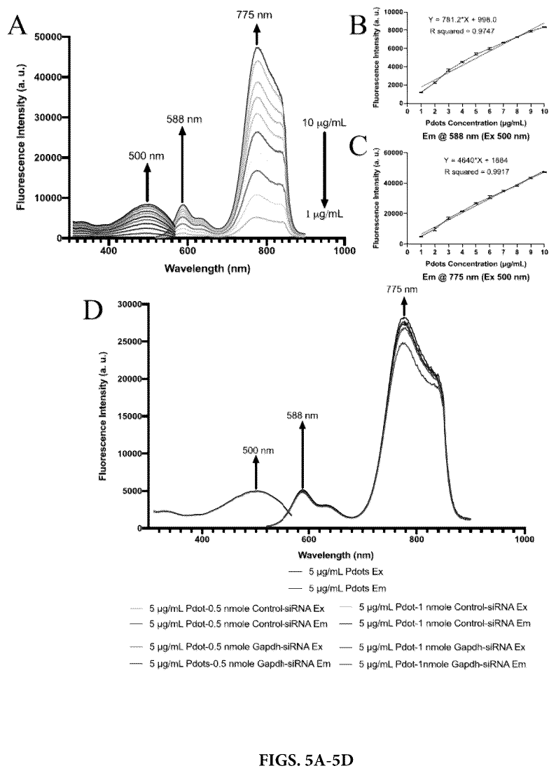

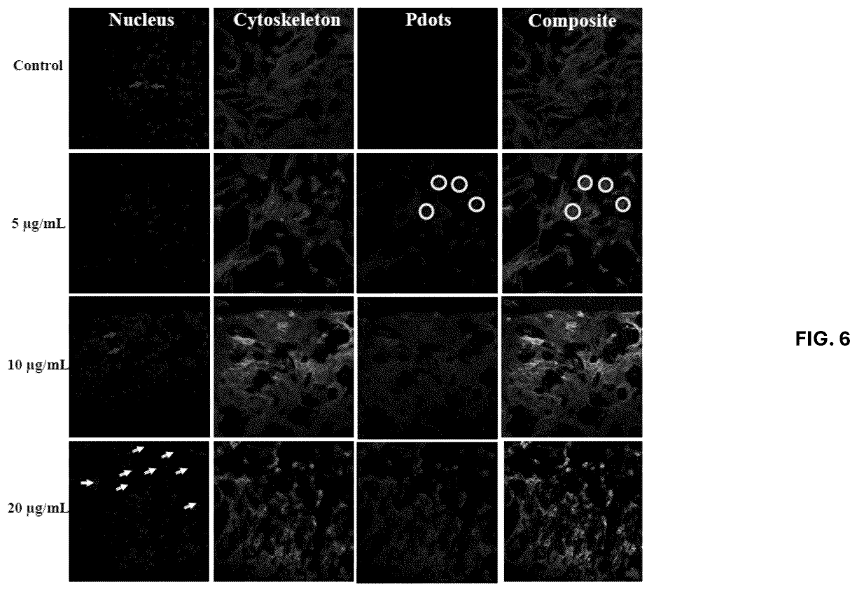

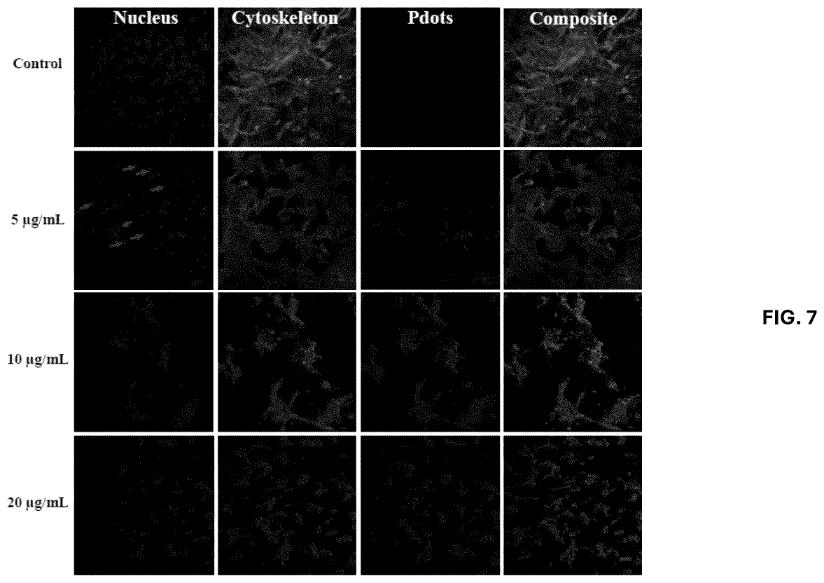

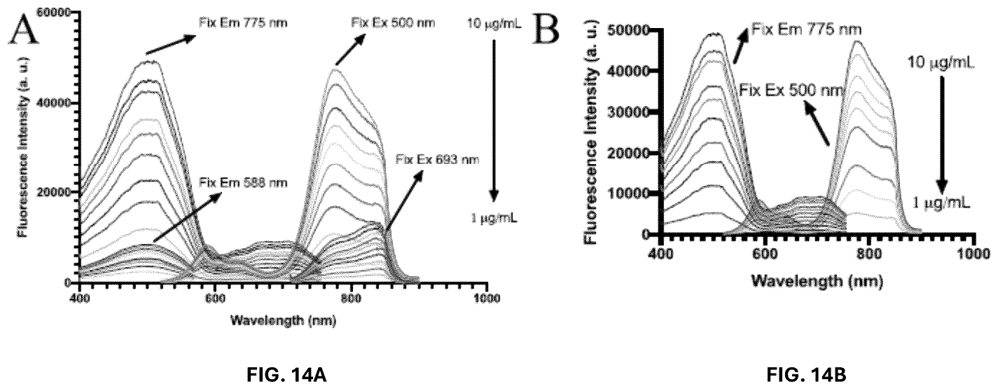

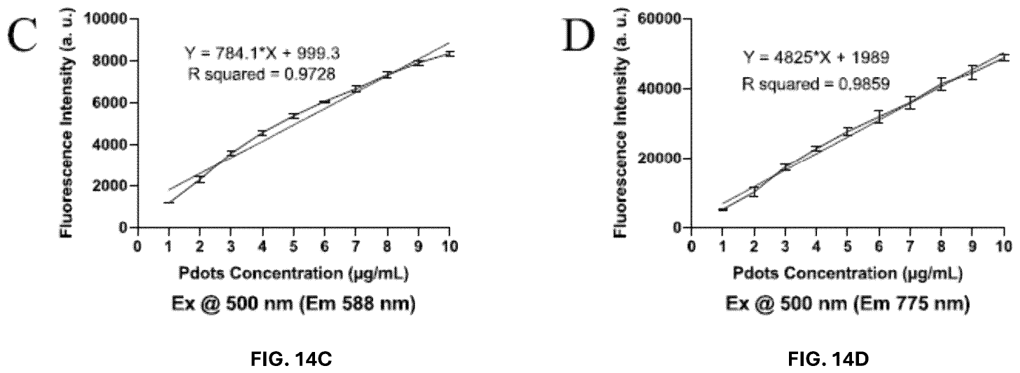

Fluorescence excitation and emission of Pdots under different concentrations are shown in FIG. 5. The spectra showed that Pdots had two emission peaks at 588 nm and 775 nm, with the excitation set at 500 nm (FIG. 5A). The two emission peaks were generated from MEHPPV and PCPDTBT, respectively. The absorbance result showed two absorbance peaks at 500 nm and 693 nm, respectively. The fluorescence properties of the Pdots were also investigated using 693 nm as the fixed excitation wavelength and run as an emission spectra series. When the emission was set at 775 nm, and the excitation spectra series run, the excitation peak fluorescence intensity at 500 nm had a similar intensity compared with the emission peak fluorescence intensity at 775 nm (FIG. 14). Overall, there were two excitation peaks at 500 nm and 693 nm, respectively, and two emission peaks at 588 nm and 775 nm, respectively. The two emission peaks had a good linear relationship under a concentration series of Pdots with R square equal to 0.9747 under emission peak at 588 nm and 0.9917 under emission peak at 775 nm (FIGS. 5B, 5C). It is likely that the energy transfer between MEHPPV and PCPDTBT resulted in the excitation and emission peak and intensity shift observed. This type of shift has been detected with similar polymers in previous research (34).

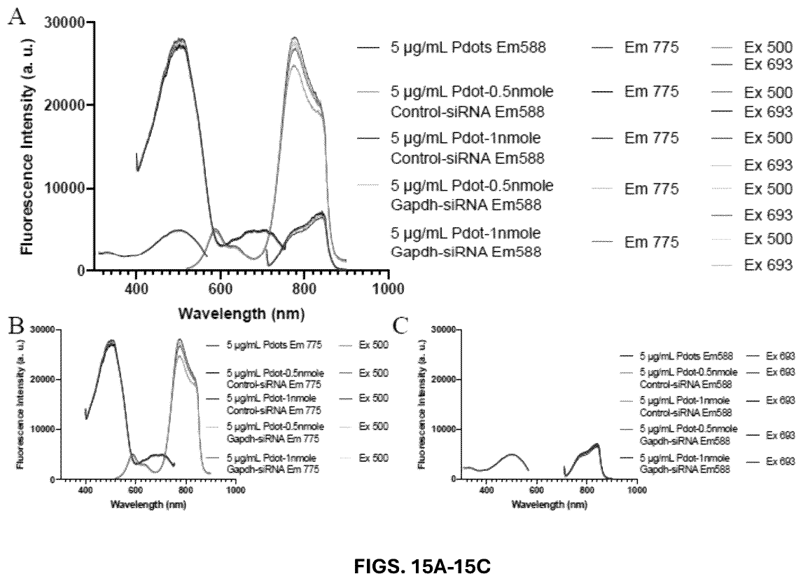

In order to determine how the binding of the siRNA affected the fluorescence properties of the Pdots, fluorescence intensity was quantified across different amounts of Pdots bound to either control siRNA or Gapdh siRNA. The Pdots bound to siRNA had similar fluorescent intensity compared to Pdots alone (FIG. 5D). Both Pdot-control siRNA and Pdot-Gapdh siRNA had two excitation peaks at 500 nm and 693 nm and two emission peaks at 588 nm and 775 nm (FIG. 15).

### Assessment of Pdots In Vitro Labeling and Cytotoxicity

To optimize cellular uptake ability and evaluate the cytotoxicity of Pdots, a rapidly dividing primary culture cell type was chosen, brain micro-vascular fibroblast (BMVFb) cells. Typically, vascular fibroblast cells are recruited to the perivascular space near endothelial cells, differentiate as pericytes and contribute to vascular stability, making them ideal for testing cytotoxicity (35). Moreover, fibroblasts are easy to grow in culture and they also have the ability to produce factors that facilitate endothelial cell (EC) growth and maturation (36). BMVFb were plated at a density of 2,500 cells/well in 8-well chamber slides in a time course series of treatment times (4, 8, 12, and 24 h) and differing Pdots concentrations (20, 10, 5, and 0 μg/mL) to determine optimal uptake and minimal labeling dose requirements. After the different Pdots treatment time periods, the medium was switched back to normal growth medium and the cells were fixed, washed, labeled and imaged as described above.

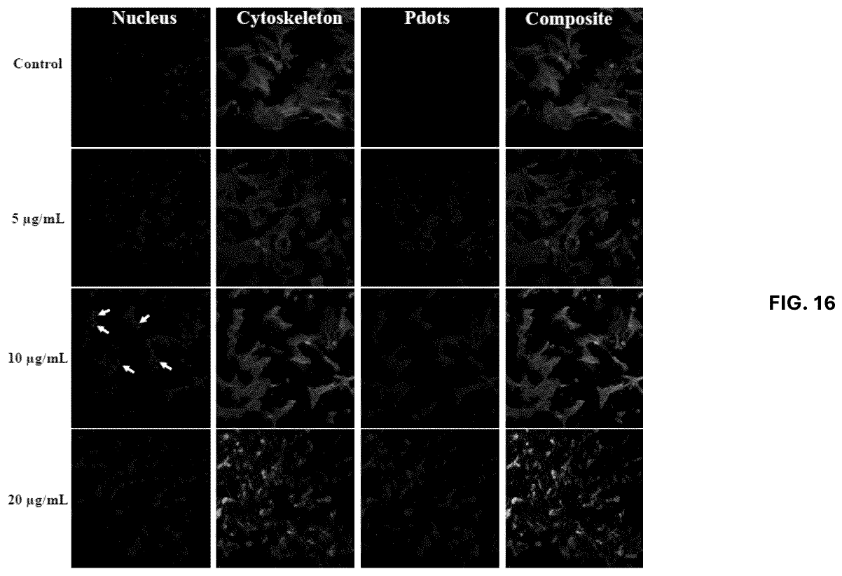

The Pdots were readily taken up by the BMVFb (red) and easily visualized in comparison with the actin cytoskeleton (green) and the nuclei (blue) after a 4 h exposure time (FIG. 6) with the brightness of the Pdots fluorescence increased at the higher concentrations of Pdots (compare 5 to 20 μg/mL). The BMVFb cells incubated without Pdots had no signal in the red channel (control panels). The red arrows highlight select cells with clear, round nuclei with the Pdots fluorescent signal localized to the perinuclear region of the cytoplasm (yellow circles). Treatment of the BMVFb cells with 20 μg/mL Pdots resulted in numerous cells with small, round or condensed and pyknotic nuclei (white arrows). The BMVFb cells incubated with Pdots for 8 h under different concentrations showed a similar phenomenon (FIG. 16); however, the small, round and pyknotic nuclei were more abundant in the cultures treated with 10 μg/mL Pdots suggesting increased cellular sensitivity with a longer exposure to Pdots.

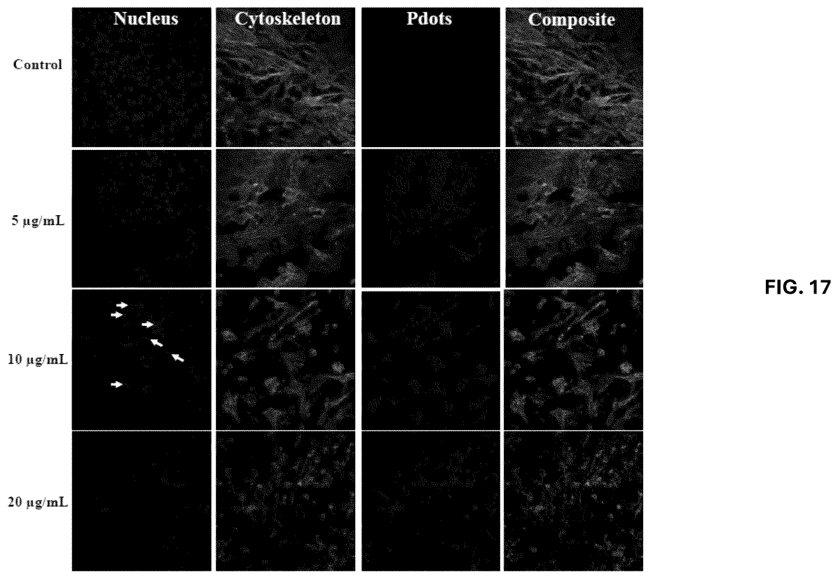

Similar results were observed after an 12 h treatment window, with the numbers of dead (pyknotic nuclei) and dying (small, round nuclei) cells increasing. In contrast, cells appeared healthy after treatment with 5 μg/mL Pdots (FIG. 17). When BMVFb cells were incubated with different concentration of Pdots for 24 h the intermediate and pyknotic nuclei were substantially increased at the 5 μg/mL dose. The dead cells increased at 10 μg/mL with almost 100% toxicity at the 20 μg/mL dose (FIG. 7).

### Cell Viability Under Different Incubation Times and Concentrations of Pdots

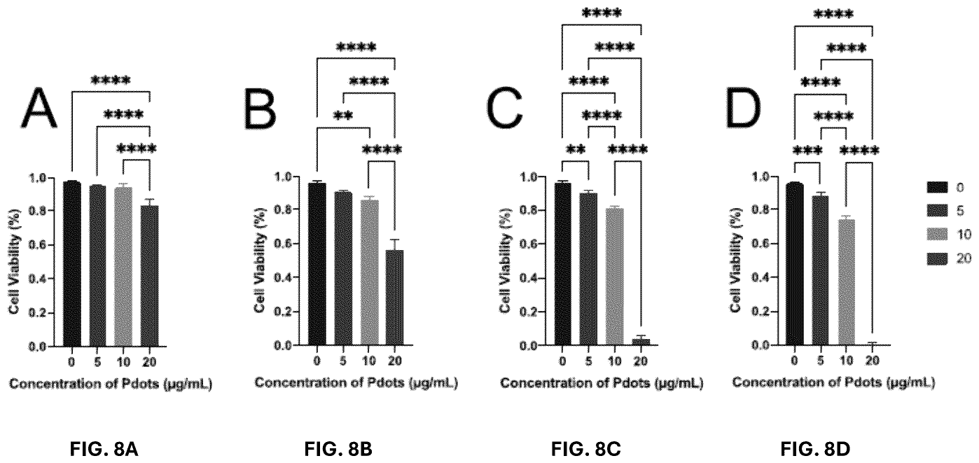

Live and dead cells were recognized based on the shape of nuclei under various incubation times and concentrations. As FIG. 8A shows, Pdots had no significant toxicity lower than 10 μg/mL, and the percentage of cell viability was 83.3% under the highest concentration at 20 μg/mL. Pdots showed less toxicity under high concentration at 20 μg/mL with short incubation time at four h. With increasing incubation time, cell toxicity increased, and cell viability was 56.3% and 85.8% at 20 μg/mL and 10 μg/mL, respectively, for the 8 h incubation period (FIG. 8B). Cell toxicity was increased at 20 μg/mL with 12 h incubation with cell viability dropping to 4.1%, in contrast to the cell viability at 81.3% with 10 μg/mL treatment (FIG. 8C). After the cells were incubated with Pdots for 24 h, the majority of the cells were dead with 20 μg/mL treatment (FIG. 8D).

Overall, 4 h incubation of BMVFb with Pdots was an ideal time for cell uptake with the least amount of toxicity. The fluorescent signal from the Pdots was distributed around the nuclei in the cytoplasm but was not detected inside the nuclei. At a dose of 20 μg/mL, the Pdots were always toxic, and 10 μg/mL of Pdots showed toxicity at 8 h treatment and beyond. 5 μg/mL of Pdots were minimally toxic, but the fluorescent signal was not as bright as that of the Pdots at 10 μg/mL. The 10 μg/mL of Pdots at 4 h showed the optimal combination of minimal toxicity with strong fluorescence intensity.

### Determining the Duration of Pdots Fluorescent Signal in Cultured Cells

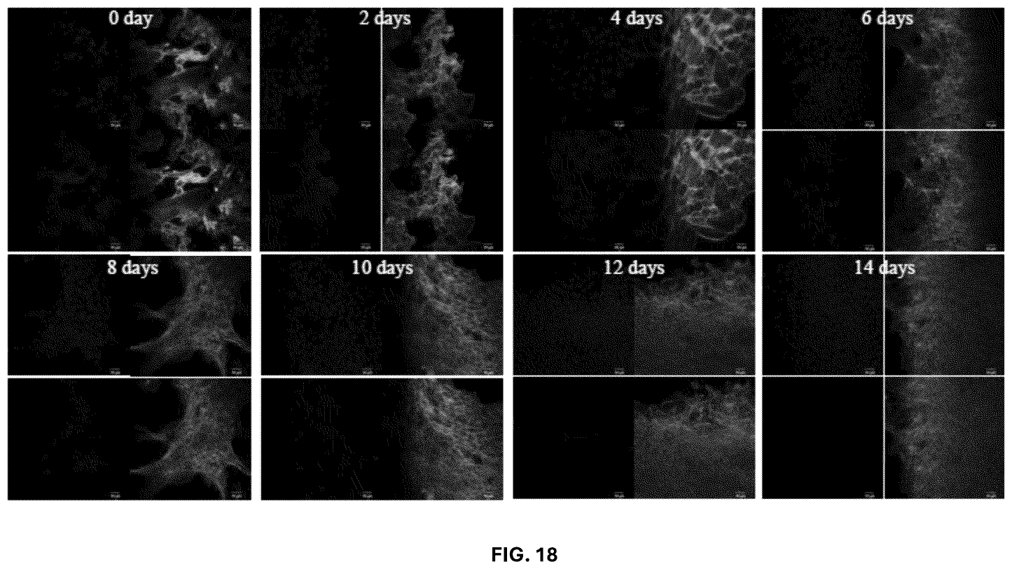

Since the incubation time and different concentrations of Pdots with BMVFb had been optimized, it was determined how long the Pdots fluorescent signal were persisting over time in culture. After a 4 h incubation with 10 μg/mL of Pdots, the BMVFb were transitioned back to normal growth medium and maintained for 14 days. The day of plating was considered day 0 for the time series with time points collected every two days thereafter. The cells were labeled with DAPI to bind to the minor groove of DNA in the nuclei and Alexa Fluor 488 phalloidin to bind to the actin cytoskeleton to visualize the relative location of Pdots in the cells. Confocal images were captured from different time points (FIG. 18). From 0-day until 8-days, the fluorescent signal from the Pdots (red channel) was readily detected in the cells. Interestingly, as the cells underwent normal division, the fluorescent Pdots signal was dispersed to the daughter cells and remained visible throughout the culture. A weak fluorescent signal was detected up until 10 days in culture. This result suggests that Pdots can be applied as a tool for siRNA delivery and can also be tracked regarding their long-term fluorescent signal in cultured cells.

### Determining Cellular Uptake for Pdot-siRNA in BMVEC

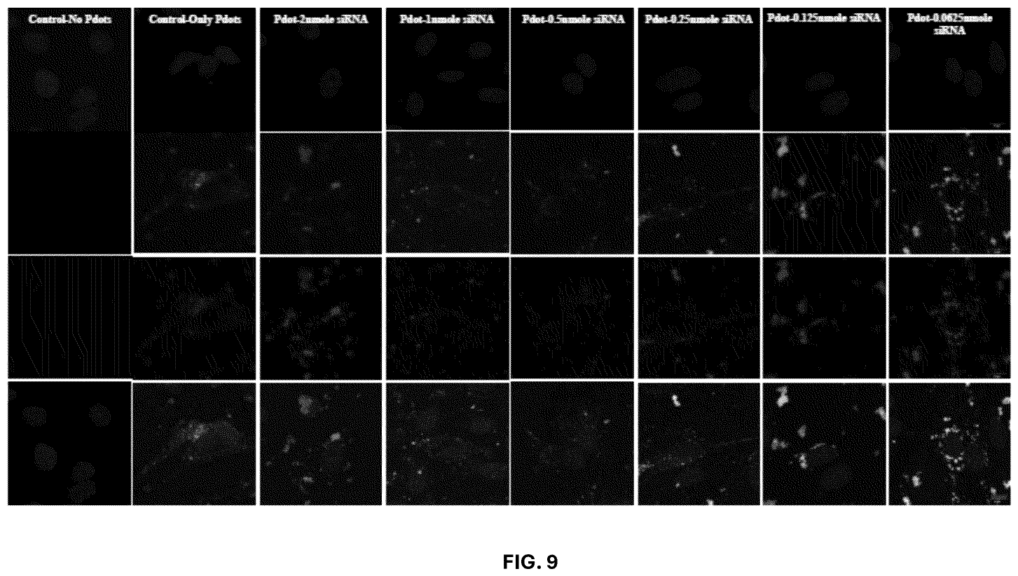

While BMVFb cell images provided a unique opportunity to test the biocompatibility of the cell with Pdots in a rapidly dividing population, BMVEC provide another vascular cell population in which to test this novel tool. The vascular endothelium is critical in regulating the interaction of circulating cells with the blood vessel wall. Vascular EC imaging can provide information about the vasculature's permeability and or the vessels' functional abnormalities. To expand the type of cell that was tested for bioimaging and to investigate the amount of Pdot-siRNA that can be taken up by the cells, the Pdot-siRNA was deployed in BMVEC. Pdots loaded with different control siRNA amounts were incubated with BMVEC for 4 h. The cells were fixed and immunolabeled with DAPI and then imaged using an Olympus BX51WI fluorescence microscope. As shown in FIG. 9, the BMVEC incubated without Pdots showed no fluorescence in the cytoplasm as expected. BMVEC incubated with Pdots displayed the expected emission (green fluorescence, detection range 550-650 nm) as well as NIR (red channel, detection range 700-800 nm) imaging pattern since Pdots have two emission peaks at 588 nm and 775 nm. Pdots with different amounts of control siRNA, including 2nmole, 1 nmole, 0.5 nmole, 0.25 nmole, 0.125 nmole, and 0.0625 nmole, displayed a similar cytoplasmic labeling pattern when compared to cells treated with Pdots alone as was observed with BMVFb. Immunolabeling results verified that the Pdot-siRNA provided a robust signal, localized primarily around the nucleus and throughout the cytoplasm for BMVEC.

### Investigation of Gapdh Target Gene Expression

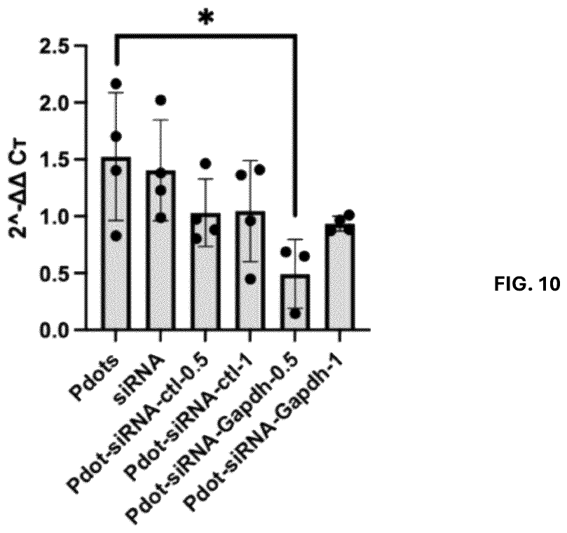

In order to determine if the Pdot-Gapdh siRNA is able to inhibit the expression of the target gene, Gapdh, changes in transcript (FIG. 10) and protein volume (FIG. 11) were assessed. BMVEC were plated in 24-well cell culture plates at a density of 50,000 cells/well and allowed to adhere for 24 h. Cells were then treated with Pdots without siRNA, Pdots-Control siRNA, and Pdot-Gapdh siRNA at two different concentrations for 4 h. Cells were transitioned to complete DMEM for an additional 20 h prior to collecting total RNA as described above. Changes in Gapdh gene expression relative to TATA Binding Protein (TBP) as a reference gene were calculated using the AA Cr method and the resulting ratio graphed as the mean and standard deviation from the mean (FIG. 10). Values were log 10 transformed prior to statistical analysis using ANOVA and Dunnett's post hoc test to determine overall differences across the population means and conduct pair-wise comparisons across conditions, respectively. Analysis reviewed a statistically significant difference in the Gapdh/TBP ratio across the means and Dunnett's multiple comparison's test indicated that Pdot-Gapdh siRNA was significantly different from the control. Gapdh expression was detected in both control conditions but reduced in the cells treated with Pdot-Gapdh siRNA relative to Pdots alone.

To assess Pdot-Gapdh siRNA effects at the protein level, the same treatment paradigm was conducted for cultures set up in parallel to immunolabeling for Gapdh protein (FIG. 11). The Gapdh was detected throughout the cytoplasm of the cells and pronounced in the perinuclear region (green channel) in both control conditions (Pdots, Pdots-Control siRNA; left panels). The labeling intensity was reduced in cultures treated with Pdot-Gapdh siRNA (FIG. 11, right panels). Pdot detection in two distinct emission spectra, 550-650 nm (red violet channel) and NIR 700-800 nm (red channel), were consistent with previous results.

### CONCLUSIONS

Provided herein is a novel nanotool was successfully developed: the Pdot-based nanoplatform for siRNA delivery. Positively charged Pdots with green (588 nm) and NIR (775 nm) fluorescence were synthesized and bound to negatively charged Control or Gapdh siRNA. Absorbance and surface properties of the Pdot-siRNA verified that siRNA was successfully conjugated with Pdots and the Pdots were readily taken up by brain-derived vascular cells. Further, the Pdot-siRNA had bright, stable fluorescence in two distinct emission wavelengths and the bound siRNA was able to inhibit target gene expression. A wide range of nanomaterials have been used to deliver small molecules, siRNA, and other proteins to cells (37,38); however, limitations in terms of toxicity, specificity or off-loading have remained a barrier to utilizing these tools more broadly. The novel Pdot-siRNA nanotool described herein represents a clear advantage for conducting dual imaging and gene expression inhibition in a variety of culture systems. The fact that the gene expression was successfully inhibited at the transcript and protein levels indicates that the Pdot-siRNA were taken up by the cells as a complex and that the siRNA was able to bind to its complementary target. This offers the benefit of visualizing which cells have taken up the Pdot and the accompanying siRNA, further refining target-specific assessment and outcomes.

Although Gapdh was used as a proof-of-principle target in the current study, there are broader implications with regard to using this approach for other targets or for regulation of the Gapdh gene expression, itself. Gapdh has been widely identified as playing a key role energy metabolism based on its ability to generate glyceraldehyde-3-phosphate and its contribution to a variety of cell processes (39). Indeed, increased Gapdh gene expression and enzymatic function has been correlated with tumor cell proliferation owing to its enzymatic function for production of ATP and pyruvate under hypoxic microenvironmental conditions (40,41). By controlling Gapdh gene expression as a potential regulator of tumor cell death, this can serve as a new avenue for therapeutic targets. One possible application is via targeting the Gapdh in tumor vasculature. Most solid tumor cancers and their metastases are highly dependent on the structure, function or angiogenesis of blood vessels in and around the tumor. In a recent study with vascular EC, expression of the GAPDH gene was markedly increased under hypoxia stress (40). The results of the current study lend support to the idea that target-specific siRNA can be delivered to cells, in this case Gapdh. Visualization can also be achieved where and when the siRNA has been taken up. This type of dual-functional nanomaterial offers the advantage of imaging and targeted inhibition of gene expression in one small package.

### SUMMARY

Polymers are one of the most promising materials for siRNA and anti-cancer drug delivery. Cationic polymer, such as polyethyleneimine (PEI), chitosan, polyamidoamine (PAMAM) dendrimers, and polylysine are commonly used for delivery applications. Amphiphilic polymers, such as polystyrene (PS) and polystyrene graft ethylene oxide functionalized with carboxyl groups (PS-PEG-COOH), polylactic acid-co-glycolic acid (PLGA), 1,2-distearoyl-sn-glycero-3-phosphoethanolamine-N-[carboxy (polyethylene glycol), NHS ester] (DSPE-PEG-NHS), and functionalized DSPE-PEG-PEI have been employed both as polymer nanoparticles backbone and support hydrophilic functional groups. However, payload delivery depends on the interaction between surface functional groups of polymers and drugs. Additionally, most of these polymer nanoparticles only provide a nanoplatform without fluorescence indication.

Generally, liposomes, and inorganic and polymers materials have served as biomolecule delivery to control payload release (56-67). Due to their biocompatibility and the encapsulating capacity for biomolecular agents, liposomes have been well applied in preclinical studies as drug and imaging agent carriers. The challenge is stability for in vivo treatment, which seriously affects the delivery efficiency of loaded drugs. Moreover, different types of liposomal nanoplatforms in clinical trials are more difficult than conventional liposomal types. There are no versatile and stable liposomal nanoplatforms suitable for all the in vivo microenvironments. Therefore, more investigations are necessary to expand the desirable aspects as a drug delivery system.

Inorganic materials-based nanoparticles have also been investigated for biomedical delivery system, such as carbon nanotubes, iron oxide nanoparticles, mesoporous silica nanoparticles, gold nanoparticles. The advantages of these nanoplatform are their stability, easy surface modification and bioconjugation. Although these inorganic nanoplatforms have been widely investigated and shown their potential as delivery vehicles for anticancer agents or siRNA, most of these materials have not been verified for their clinical toxicity. There is not enough evidence to see in vivo toxicity and delivery efficiency for theranostic purposes. Further toxicity evaluation is needed to investigate.

Semiconducting polymer dots (Pdots) have been developed as fluorescent nanomaterials for bioanalysis and bioimaging because of their outstanding properties, such as high fluorescence intensity, excellent biocompatibility with lower toxicity, outstanding photostability for long-term tracking and monitoring, and easy surface modification to increase the solubility (42-45). Dual emissive fluorescent nanoplatform gives cellular images with two different colors, one is visible window fluorescence, another one is near-infrared fluorescence (NIRF). Compared with single-emissive fluorescence, the benefit of dual emissive fluorescence is that the site of interest can be easily distinguished from the false positive signal generated by accidental fluorescent biomolecules during the monitoring process, avoid environmental influence and improve signal-to-noise ratio (46-48). NIRF also shows more attractive benefits than any other ultraviolet and visible window fluorescence because of their low auto-fluorescence and absorption of radiation, and less light scattering (49-51). In addition to fluorescence imaging, Pdots have also been developed for a wide variety of biomedical applications, such as photodynamic therapy (PDT), photothermal therapy (PTT) (52-55). Due to the high flexibility of the polymer matrix, Pdots are also considered an ideal delivery platform for therapeutic agents, such as conventional drug delivery and nucleic acid (DNA plasmid or siRNA) delivery (44, 52, 56, 57). These biomaterials made the nanoplatform a good candidate for long-term in vivo imaging and for targeted, deep tissue penetration. Delivery can be achieved without ionizing radiation. Additionally, this platform provides good spatial and temporal resolution, thereby providing precise guidance for therapy.

### BIBLIOGRAPHY

1. Ramjiawan, R. R.; Griffioen, A. W.; Duda, D. G. Angiogenesis 2017, 20 (2), 185-204. DOI: 10.1007/s10456-017-9552-y.
2. Debela, D. T.; Muzazu, S. G.; Heraro, K. D.; Ndalama, M. T.; Mesele, B. W.; Haile, D. C.; Kitui, S. K.; Manyazewal, T. SAGE Open Med 2021, 9, 20503121211034366. DOI: 10.1177/20503121211034366 From NLM.
3. Gupta, S. L.; Basu, S.; Soni, V.; Jaiswal, R. K. Mol Biol Rep 2022, 49 (10), 9903-9913. DOI: 10.1007/s11033-022-07525-8 From NLM.
4. Li, T.; Kang, G.; Wang, T.; Huang, H. Oncol. Lett. 2018, 16 (1), 687-702. DOI: 10.3892/ol.2018.8733.
5 Chen, X.; Mangala, L. S.; Rodriguez-Aguayo, C.; Kong, X.; Lopez-Berestein, G.; Sood, A. K. Cancer Metastasis Rev 2018, 37 (1), 107-124. DOI: 10.1007/s10555-017-9717-6 From NLM.
6. Kim, B.; Park, J.-H.; Sailor, M. J. Adv. Mater. 2019, 31 (49), 1903637. DOI: 10.1002/adma.201903637 (accessed 2020 Sep. 7).
7. Xu, W.; Jiang, X.; Huang, L. 5.42-RNA Interference Technology. In Comprehensive Biotechnology (Third Edition), Moo-Young, M. Ed.; Pergamon, 2019; pp 560-575.
8. Wang, J.; Lu, Z.; Wientjes, M. G.; Au, J. L. Aaps j 2010, 12 (4), 492-503. DOI: 10.1208/s12248-010-9210-4 From NLM.
9. Sato, Y.; Matsui, H.; Sato, R.; Harashima, H. J Control Release 2018, 284, 179-187. DOI: 10.1016/j.jconrel.2018.06.017 From NLM.
10. Chen, Z.; Krishnamachary, B.; Pachecho-Torres, J.; Penet, M.-F.; Bhujwalla, Z. M. WIRE Nanomed. Nanobio. 2020, 12 (2), e1595. DOI: 10.1002/wnan.1595.
11. Li, Y.; Ji, T.; Torre, M.; Shao, R.; Zheng, Y.; Wang, D.; Li, X.; Liu, A.; Zhang, W.; Deng, X.; et al. Nat. Commun. 2023, 14 (1), 6659. DOI: 10.1038/s41467-023-41946-8.
12. Seynhaeve, A. L. B.; Dicheva, B. M.; Hoving, S.; Koning, G. A.; ten Hagen, T. L. M. Journal of Controlled Release 2013, 172 (1), 330-340. DOI: https://doi.org/10.1016/j.jconrel.2013.08.034.
13. Yang, K.; Tran, K.; Salvati, A. Biomolecules 2023, 13 (1), 59.
14. Bharti, C.; Nagaich, U.; Pal, A. K.; Gulati, N. Int J Pharm Investig 2015, 5 (3), 124-133. DOI: 10.4103/2230-973x.160844 From NLM.
15. Ajith, S.; Almomani, F.; Elhissi, A.; Husseini, G. A. Heliyon 2023, 9 (11), e21227. DOI: 10.1016/j.heliyon.2023.e21227 From NLM.
16. Thomas, T. J.; Tajmir Riahi, H. A.; Pillai, C. K. S. Molecules 2019, 24 (20), 3744. DOI: 10.3390/molecules24203744 PubMed.
17. Xia, W.; Tao, Z.; Zhu, B.; Zhang, W.; Liu, C.; Chen, S.; Song, M. Int. J. Mol. Sci. 2021, 22 (17), 9118.
18. Zhu, H.; Li, J.; Qi, X.; Chen, P.; Pu, K. Nano Lett. 2018, 18 (1), 586-594. DOI: 10.1021/acs.nanolett.7b04759.
19. Lyu, Y.; Zeng, J.; Jiang, Y.; Zhen, X.; Wang, T.; Qiu, S.; Lou, X.; Gao, M.; Pu, K. ACS Nano 2018, 12 (2), 1801-1810. DOI: 10.1021/acsnano.7b08616.
20. Zhou, R.; Zheng, Y.; Qian, L.; Yang, Y.; Holloway, P. H.; Xue, J. Nanoscale 2012, 4 (11), 3507-3514, 10.1039/C2NR30210A. DOI: 10.1039/C2NR30210A.
21. Deng, Z.; Qiao, G.; Ma, L.; Zhang, Q.; Zhang, P.; Cui, D. ACS Appl. Nano Mater. 2021, 4 (12), 13523-13533. DOI: 10.1021/acsanm.1c02929.
22. Darland, D. C.; Cain, J. T.; Berosik, M. A.; Saint-Geniez, M.; Odens, P. W.; Schaubhut, G. J.; Frisch, S.; Stemmer-Rachamimov, A.; Darland, T.; D'Amore, P. A. Dev. Biol. 2011, 358 (1), 9-22. DOI: https://doi.org/10.1016/j.ydbio.2011.06.045.
23. Cain, J. T.; Berosik, M. A.; Snyder, S. D.; Crawford, N. F.; Nour, S. I.; Schaubhut, G. J.; Darland, D. C. Dev. Neurobiol. 2014, 74 (1), 63-81. DOI: https://doi.org/10.1002/dneu.22130.
24. Wu, C.; Bull, B.; Szymanski, C.; Christensen, K.; McNeill, J. ACS Nano 2008, 2 (11), 2415-2423. DOI: 10.1021/nn800590n.
25. Rasband, W. ImageJ, U.S. National Institutes of Health, Bethesda, Maryland, USA. 2011.
26. Rao, X.; Huang, X.; Zhou, Z.; Lin, X. Biostat Bioinforma Biomath 2013, 3 (3), 71-85. From NLM.
27. Perche, F.; Patel, N. R.; Torchilin, V. P. Journal of Controlled Release 2012, 164 (1), 95-102. DOI: https://doi.org/10.1016/j.jconrel.2012.09.003.
28. Kohay, H.; Sarisozen, C.; Sawant, R.; Jhaveri, A.; Torchilin, V. P.; Mishael, Y. G. Acta Biomaterialia 2017, 55, 443-454. DOI: https://doi.org/10.1016/j.actbio.2017.04.008.
29. Tian, G.; Pan, R.; Zhang, B.; Qu, M.; Lian, B.; Jiang, H.; Gao, Z.; Wu, J. Frontiers in Pharmacology 2019, 10, Original Research. DOI: 10.3389/fphar.2019.00004.
30. Wu, C.; Chiu, D. T. Angew. Chem. Int. Ed. 2013, 52 (11), 3086-3109. DOI: 10.1002/anie.201205133 (accessed 2020 Oct. 2).
31. Wu, X.; Chiu, D. T. In Conjugated Polymers for Biological and Biomedical Applications, 2018; pp 59-85.
32. Gupta, N.; Chan, Y.-H.; Saha, S.; Liu, M.-H. ACS Applied Polymer Materials 2020, 2 (10), 4195-4221. DOI: 10.1021/acsapm.0c00629.
33. Patel, S.; Kim, J.; Herrera, M.; Mukherjee, A.; Kabanov, A. V.; Sahay, G. Adv Drug Deliv Rev 2019, 144, 90-111. DOI: 10.1016/j.addr.2019.08.004 From NLM.
34. Ozel, I. O.; Ozel, T.; Demir, H. V.; Tuncel, D. Opt. Express 2010, 18 (2), 670-684. DOI: 10.1364/OE.18.000670.
35. Rajan, A. M.; Ma, R. C.; Kocha, K. M.; Zhang, D. J.; Huang, P. PLOS Genet 2020, 16 (10), e1008800. DOI: 10.1371/journal.pgen. 1008800 From NLM.
36. Caneparo, C.; Baratange, C.; Chabaud, S.; Bolduc, S. Scientific Reports 2020, 10 (1), 9291. DOI: 10.1038/s41598-020-66145-z.
37. Mitchell, M. J.; Billingsley, M. M.; Haley, R. M.; Wechsler, M. E.; Peppas, N. A.; Langer, R. Nature Reviews Drug Discovery 2021, 20 (2), 101-124. DOI: 10.1038/s41573-020-0090-8.
38. Luther, D. C.; Huang, R.; Jeon, T.; Zhang, X.; Lee, Y.-W.; Nagaraj, H.; Rotello, V. M. Advanced Drug Delivery Reviews 2020, 156, 188-213. DOI:
   https://doi.org/10.1016/j.addr.2020.06.020.
39. Bruns, G. A. P.; Gerald, P. S. Science 1976, 192 (4234), 54-56. DOI: doi: 10.1126/science.176725.
40. Graven, K. K.; Troxler, R. F.; Kornfeld, H.; Panchenko, M. V.; Farber, H. W. Journal of Biological Chemistry 1994, 269 (39), 24446-24453. DOI: https://doi.org/10.1016/S0021-9258 (19) 51104-8.
41. Nicholls, C.; Li, H.; Liu, J.-P. Clinical and Experimental Pharmacology and Physiology 2012, 39 (8), 674-679. DOI: https://doi.org/10.1111/j.1440-1681.2011.05599.x.
42. Wu, C.; Bull, B.; Szymanski, C.; Christensen, K.; McNeill, J. ACS Nano 2008, 2 (11), 2415-2423.
43. Wu, C.; Schneider, T.; Zeigler, M.; Yu, J.; Schiro, P. G.; Burnham, D. R.; McNeill, J. D.; Chiu, D. T. Journal of the American Chemical Society 2010, 132 (43), 15410-15417.
44. Wu, C.; Chiu, D. T. Angewandte Chemie International Edition 2013, 52 (11), 3086-3109.
45. Wu, X.; Chiu, D. T., Conjugated Polymers for Biological and Biomedical Applications, 2018; pp 59-85.
46. Ren, M.; Deng, B.; Wang, J.; Liu, Z.; Lin, W., Journal of Materials Chemistry B 2015, 3 (33), 6746-6752.
47. Dai, C.; Yang, C.; Yan, X., Analytical Chemistry 2015, 87 (22), 11455-11459.
48. Kukhta, N. A.; Bryce, M. R., Dual emission in purely organic materials for optoelectronic applications. Materials Horizons 2021, 8 (1), 33-55.
49. Amiot, C. L.; Xu, S.; Liang, S.; Pan, L.; Zhao, J. X., Sensors (Basel) 2008, 8 (5), 3082-3105.
50. Escobedo, J. O.; Rusin, O.; Lim, S.; Strongin, R. M., Current Opinion in Chemical Biology 2010, 14 (1), 64-70.
51. Crosignani, V.; Dvornikov, A.; Aguilar, J. S.; Stringari, C.; Edwards, R.; Mantulin, W. W.; Gratton, E., J Biomed Opt 2012, 17 (11), 116023-116023.
52. Tang, Y.; Chen, H.; Chang, K.; Liu, Z.; Wang, Y.; Qu, S.; Xu, H.; Wu, C., ACS Applied Materials & Interfaces 2017, 9 (4), 3419-3431.
53. Shi, H.; Wang, Y.; Huang, X.; Liang, P.; Tang, Y.; Zhang, Y.; Fu, N.; Huang, W.; Dong, X., Journal of Materials Chemistry B 2018, 6 (45), 7402-7410.
54. Zhu, H.; Li, J.; Qi, X.; Chen, P.; Pu, K., Nano Letters 2018, 18 (1), 586-594.
55. Lyu, Y.; Zeng, J.; Jiang, Y.; Zhen, X.; Wang, T.; Qiu, S.; Lou, X.; Gao, M.; Pu, K., ACS Nano 2018, 12 (2), 1801-1810.
56. Wei, L.; Zhang, D.; Zheng, X.; Zeng, X.; Zeng, Y.; Shi, X.; Su, X.; Xiao, L., Nanotheranostics 2018, 2 (2), 157-167.
57. Wang, F.; Chen, H.; Liu, Z.; Mi, F.; Fang, X.; Liu, J.; Wang, M.; Lo, P. K.; Li, Q., New Journal of Chemistry 2019, 43 (36), 14443-14449.
58. Barrett, S. E.; Guidry, E. N., SiRNA Delivery Methods: Methods and Protocols, Shum, K.; Rossi, J., Eds. Springer New York: New York, NY, 2016; pp 11-25.
59. Hasan, M. T.; Campbell, E.; Sizova, O.; Lyle, V.; Akkaraju, G.; Kirkpatrick, D. L.; Naumov, A. V., Cancers 2019, 11 (8), 1175.
60. Li, Y.; Huang, X.; Lee, R. J.; Qi, Y.; Wang, K.; Hao, F.; Zhang, Y.; Lu, J.; Meng, Q.; Li, S.; Xie, J.; Teng, L., Molecules 2016, 21 (10), 1314.
61. Liu, Y.; Gunda, V.; Zhu, X.; Xu, X.; Wu, J.; Askhatova, D.; Farokhzad, O. C.; Parangi, S.; Shi, J., Proceedings of the National Academy of Sciences 2016, 113 (28), 7750-7755.
62. Siu, K. S.; Zheng, X.; Liu, Y.; Zhang, Y.; Zhang, X.; Chen, D.; Yuan, K.; Gillies, E. R.; Koropatnick, J.; Min, W.-P., Bioconjugate Chemistry 2014, 25 (10), 1744-1751.
63. Xie, M.; Zhu, Y.; Xu, S.; Xu, G.; Xiong, R.; Sun, X.; Liu, C., Nanoscale 2020, 12 (21), 11497-11509.
64. Meng, H.; Liong, M.; Xia, T.; Li, Z.; Ji, Z.; Zink, J. I.; Nel, A. E., ACS Nano 2010, 4 (8), 4539-4550.
65. Ma, X.; Zhao, Y.; Ng, K. W.; Zhao, Y., Chemistry-A European Journal 2013, 19 (46), 15593-15603.
66. Hassan, M. A.; Khan, A. Z.; Sajid, M. M.; Javed, Y.; Ullah, A.; Shad, N. A.; Sharma, S. K.; Shafique, M.; Sarwar, M., Spinel Nanoferrites: Synthesis, Properties and Applications, Sharma, S. K., Ed. Springer International Publishing: Cham, 2021; pp 101-128.
67. Babu, A.; Munshi, A.; Ramesh, R., Drug Development and Industrial Pharmacy 2017, 43 (9), 1391-1401.

All publications, patents, and patent applications, Genbank sequences, websites and other published materials referred to throughout the disclosure herein are herein incorporated by reference to the same extent as if each individual publication, patent, or patent application, Genbank sequences, websites and other published materials was specifically and individually indicated to be incorporated by reference. In the event that the definition of a term incorporated by reference conflicts with a term defined herein, this specification shall control.

What is claimed is:

1. A polymer dot-nucleic acid composition comprising:
a polymer dot comprising poly[(2-methoxy-5-(2-ethylhexyloxy)-1,4-phenylenevinylene) (MEHPPV); poly[(2,6-(4,4-bis(2-ethylhexyl)-4H-cyclopenta(2,1-b;3,4-b′)dithiophene)-alt-4,7 (2,1,3-benzothiadiazole)) (PCPDTBT); and 1,2-distearoyl-sn-glycero-3-phosphoethanolamine-polyethylene glycol-polyetherimide (DSPE-PEG-PEI); and
one or more inhibitory nucleic acids bound to the polymer dot.

2. The composition of claim 1, wherein the polymer dot has a diameter of about 50 to about 100 nm.

3. The composition of claim 1, wherein the nucleic acid is non-covalently bound to the polymer dot.

4. The composition of claim 1, wherein the polymer dot further comprises one or more functional groups selected from the group consisting of carboxylic acid, amino, mercapto, azido, alkyne, hydroxyl, and/or aldehyde groups.

5. The composition of claim 4, wherein the nucleic acid is covalently bound to the polymer dot.

6. The composition of claim 1, comprising a plurality of bound nucleic acid molecules on the surface of the polymer dot.

7. The composition of claim 1, wherein the inhibitory nucleic acid is small interfering RNA (siRNA).

8. The composition of claim 7, wherein the siRNA are at least 20 base pairs in length.

9. The composition of claim 7, wherein the siRNA is about 20 to about 25 base pairs in length.

10. The composition of claim 1, wherein the Pdot further comprises an additional therapeutic agent, such as a drug (e.g., chemotherapeutic agent).

11. A method to regulate gene expression in a eukaryotic cell comprising contacting and allowing uptake of the composition of claim 1 by the cell.

12. A method to inhibit gene expression comprising administering to a subject in need thereof the composition of claim 1.

13. The method of claim 12, wherein the subject has cancer, and the nucleic acid is specific for a cancer gene.

14. A method to visualize nucleic acids intracellularly comprising contacting cells with the composition of claim 1 and detecting the fluorescence of the polymer dot.


SEQUENCE LISTING (6 sequences)

SEQ ID NO: 1 - DNA, 27 bp, synthetic construct
  source 1..27: mol_type = other DNA
gacacggacaggattgacagattgata

SEQ ID NO: 2 - DNA, 26 bp, synthetic construct
  source 1..26: mol_type = other DNA
gttagcatgccagagtctcgttcgtt

SEQ ID NO: 3 - DNA, 27 bp, synthetic construct
  source 1..27: mol_type = other DNA
caagaaatgctgaatataatcccaagc

SEQ ID NO: 4 - DNA, 24 bp, synthetic construct
  source 1..24: mol_type = other DNA
agtctggattgttcttcactcttg

SEQ ID NO: 5 - DNA, 23 bp, synthetic construct
  source 1..23: mol_type = other DNA
gtggcaaagtggagatggttgcc

SEQ ID NO: 6 - DNA, 21 bp, synthetic construct
  source 1..21: mol_type = other DNA
gatgatgacccgtttggctcc